(19) United States
(12) Patent Application Publication
(10) Pub. No.: US 2024/0100201 A1
(43) Pub. Date: Mar. 28, 2024

(54) Labeling precursors and radiotracers for nuclear medicine diagnosis and therapy of prostate cancer-induced bone metastases

(71) Applicant: SCV-SpezialChemikalienVertrieb GmbH, Berlin (DE)

(72) Inventors: Frank RÖSCH, Berlin (DE); Tilman GRUS, Mainz (DE)

(73) Assignee: SCV-SpezialChemikalienVertrieb GmbH, Berlin (DE)

(21) Appl. No.: 18/260,775

(22) PCT Filed: Jan. 20, 2022
(86) PCT No.: PCT/EP2022/051289

§ 371 Date: Jul. 7, 2023

(30) Foreign Application Priority Data
Jan. 21, 2021 (DE) 10 2021 101 216.3

(51) Int. Cl. A61K 51/04

(52) U.S. Cl. CPC A61K 51/0489

(57) ABSTRACT

The invention relates to a labeling precursor for nuclear medicine diagnostics and theranostics that has the structure

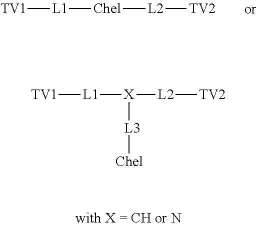

with a first PSMA-specific targeting vector TV1, a second targeting vector TV2 having osteoaffinity, a chelator Chel for complexing a radioisotope, and two or three linkers L1, L2 and L3.

The present invention relates to a labeling precursor for complexing radioactive isotopes, comprising a chelator Chel and two targeting vectors for PSMA and bone metastases conjugated with the chelator Chel.

The compounds of the invention are intended for imaging nuclear medicine diagnosis and treatment (theranostics) of bone metastases induced by prostate cancer.

### Radiotheranostics

In clinical practice, nuclear-medical diagnostic imaging methods such as positron emission tomography (PET) and single-photon emission computed tomography (SPECT) have been used to an increasing extent for about 15 years. Recently, theranostic methods have also become increasingly important.

In nuclear-medical diagnostics and therapy, tumor cells and metastases are labeled or irradiated with a radioactive isotope, for example gallium-68 (⁶⁸Ga) or lutetium-177 (¹⁷⁷Lu). Among the labeling precursors used here are those that bind coordinatively to the respective radioisotope (⁶⁸Ga, ^{99m}Tc, ¹⁷⁷Lu) and form a radiotracer. The labeling precursors comprise, as an essential chemical component, a chelator for the effective and stable complexation of the radioisotope and, as a functional component, a biological targeting vector that binds to a defined target structure in the tumor tissue. In general, the biological targeting vector has a high affinity for transmembrane receptors, proteins, enzymes or other structures of tumor cells.

After intravenous injection into the bloodstream, the radioisotope-labeled theranostic agent or radiotracer accumulates on or within the cells of the primary tumor and metastatic tissue. The aim is to deposit such a dose of radiation in the tumor that the tissue dies. At the same time, the radiation dose imparted to the healthy tissue should remain sufficiently low that damage there is tolerable.

The chelator modifies the configuration and chemical properties of the targeting vector, and generally strongly influences its affinity for tumor cells. Accordingly, the coupling between the chelator and the targeting vector is tailored in complex trial-and-error experiments or what are called biochemical screenings. This involves synthesizing a large number of labeling precursors comprising the chelator and the targeting vector, and quantifying the affinity for tumor cells in particular. The chelator and the chemical coupling to the targeting vector are crucial to the biological and nuclear-medical potency of the respective radiotheranostic.

In addition to high affinity, the labeling precursor must meet further requirements, such as
rapid and effective chelation of the respective radioisotope;
high selectivity of the final radiotheranostic for tumor cells and metastases—especially bone metastases—relative to healthy tissue;
in vivo stability, i.e. biochemical stability of the final radiotheranostic in blood serum under physiological conditions.

### Prostate Cancer

For men in industrialized countries, prostate cancer is the most common type of cancer and the third most common cause of death from cancer. In this disease, tumor growth progresses slowly. If diagnosed at an early stage, the 5-year survival rate is close to 100%. If the disease is discovered only after the tumor has metastasized, the survival rate falls dramatically. On the other hand, treating the tumor too early and too aggressively can unnecessarily impair the patient's quality of life. For example, surgical removal of the prostate can lead to incontinence and impotence. A reliable diagnosis and information about the stage of the disease are essential for successful treatment with high quality of life for the patient. A widely used diagnostic tool, in addition to palpation of the prostate by a doctor, is the determination of tumor markers in the patient's blood. The most prominent marker of prostate cancer is the concentration of prostate-specific antigen (PSA) in the blood. However, the significance of the PSA concentration is disputed, since patients with slightly elevated values often do not have prostate carcinoma, but 15% of patients with prostate carcinoma do not show elevated PSA concentration in the blood.

### Prostate-Specific Membrane Antigen (PSMA)

A target structure for the diagnosis of prostate tumors is the prostate-specific membrane antigen (PSMA). Unlike PSA, PSMA cannot be detected in blood. It is a membrane-bound glycoprotein having enzymatic activity. Its function is to cleave C-terminal glutamate from N-acetyl-aspartyl-glutamate (NAAG) and folic acid-(poly)-γ-glutamate. PSMA barely occurs in normal tissue, but is highly overexpressed by prostate carcinoma cells, with expression being closely correlated with the stage of the tumor disease. A proportion of about 40% of lymph node and bone metastases of prostate carcinomas also express PSMA.

One strategy for molecular targeting of PSMA is to bind antibodies to the protein structure of PSMA. Another approach is to use the enzymatic activity of PSMA, which is well understood. In the enzymatic binding pocket of PSMA, there are two Zn²⁺ ions that bind glutamate. In front of the center with the two Zn²⁺ ions is an aromatic binding pocket. The protein is able to expand and adapt to the binding partner (induced fit), such that it can also bind folic acid in addition to NAAG, with the pteroic acid group docking in the aromatic binding pocket. The use of the enzymatic affinity of PSMA enables uptake of the substrate into the cell (endocytosis) independently of enzymatic cleavage of the substrate.

Therefore, PSMA inhibitors in particular are of good suitability as targeting vectors for imaging diagnostic and theranostic radiopharmaceuticals or radiotracers. The radiolabeled inhibitors bind to the active site of the enzyme, but are not converted there. The bond between the inhibitor and the radioactive label is thus not broken. Favored by endocytosis, the inhibitor with the radioactive label is taken up into the cell and accumulates in the tumor cells.

Inhibitors with high affinity for PSMA (scheme 1) generally contain a glutamate motif and an enzymatically non-cleavable structure. A highly effective PSMA inhibitor is 2-phosphonomethylglutaric acid or 2-phosphonomethylpentanedioic acid (2-PMPA), in which the glutamate motif is bonded to a phosphonate group which is not cleavable by PSMA. Urea-based inhibitors form another group of PSMA inhibitors used in the clinically relevant radiopharmaceuticals PSMA-11 (scheme 2) and PSMA-617 (scheme 3).

It has been found to be advantageous to target the aromatic binding pocket of PSMA in addition to the binding pocket for the glutamate motif. For example, in the highly potent radiopharmaceutical PSMA-11, the binding motif L-lysine-urea-L-glutamate (KuE) is bound via hexyl (hexyl linker) to an aromatic HBED chelator (N,N′-bis(2-hydroxy-5-(ethylene-beta-carboxy)benzyl)ethylenediamine N,N′-diacetate).

In contrast, if L-lysine-urea-L-glutamate (KuE) is bound to the non-aromatic chelator DOTA (1,4,7,10-tetraazacyclododecane-1,4,7,10-tetraacetate), reduced affinity and accumulation in tumor tissue is observed. However, in order to able to use the DOTA chelator for a radiopharmaceutical having PSMA affinity with therapeutic radioisotopes, such as ¹⁷⁷Lu or ²²⁵Ac, the linker has to be adapted. The highly effective radiopharmaceutical PSMA-617, the current gold standard, was found by means of specific substitution of hexyl by various aromatic structures.

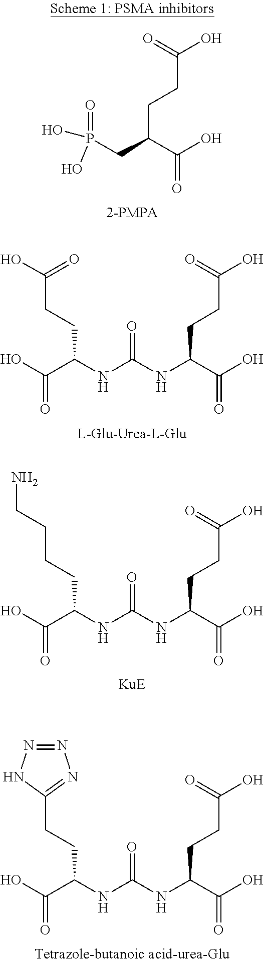 [SMILES: O=C(O)CC[C@H](CP(=O)(O)O)C(=O)O ; O=C(O)CC[C@H](NC(=O)N[C@@H](CCC(=O)O)C(=O)O)C(=O)O ; NCCCC[C@H](NC(=O)N[C@@H](CCC(=O)O)C(=O)O)C(=O)O ; O=C(O)CC[C@H](NC(=O)N[C@@H](CCc1nnn[nH]1)C(=O)O)C(=O)O]

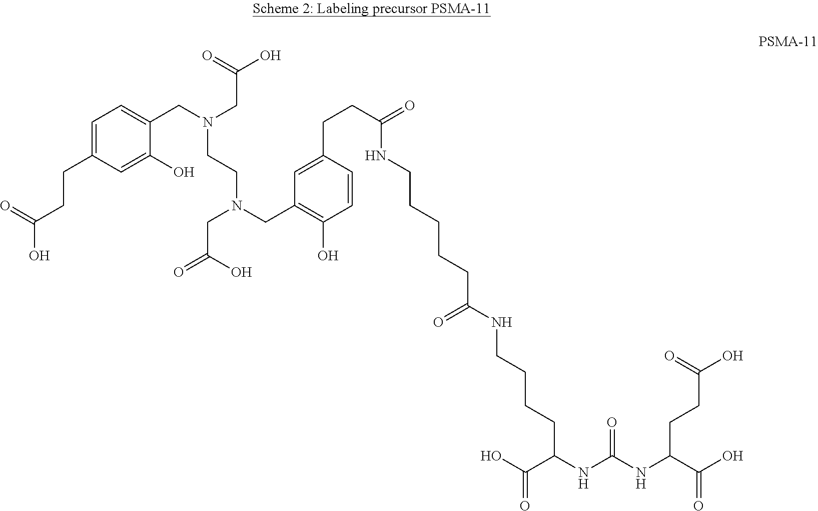 [SMILES: O=C(O)CCc1ccc(CN(CCN(CC(=O)O)Cc2cc(CCC(=O)NCCCCCC(=O)NCCCCC(NC(=O)NC(CCC(=O)O)C(=O)O)C(=O)O)ccc2O)CC(=O)O)c(O)c1]

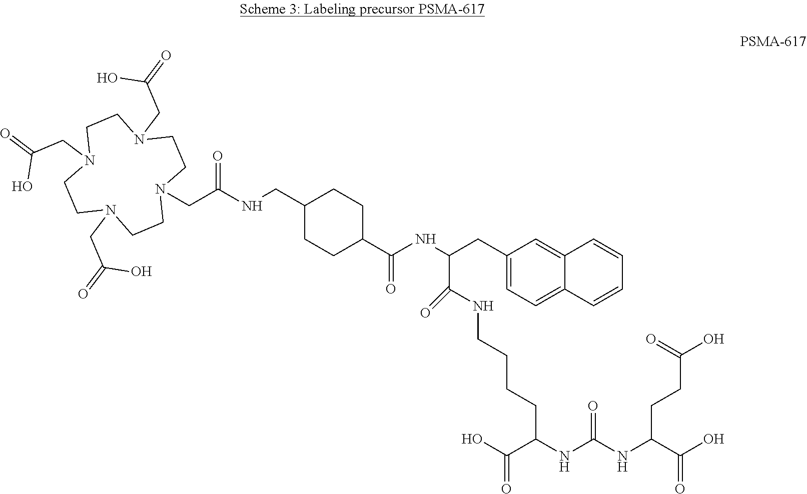 [SMILES: O=C(O)CCC(NC(=O)NC(CCCCNC(=O)C(Cc1ccc2ccccc2c1)NC(=O)C1CCC(CNC(=O)CN2CCN(CC(=O)O)CCN(CC(=O)O)CCN(CC(=O)O)CC2)CC1)C(=O)O)C(=O)O]

The prior art discloses a multitude of labeling precursors for diagnosis and theranostics of cancers with radioactive isotopes. WO 2015055318 A1 discloses radiotracers for the diagnosis and theranostics of prostate carcinomas or epithelial carcinomas, including the compound PSMA-617 shown in scheme 3.

### Bone Metastases and Bisphosphonates

Bisphosphonates (BP) are used in clinical practice for the treatment of disorders of bone metabolism and calcium metabolism. These include Paget's disease, osteoporosis and the conventional systemic therapy of bone tumors. Bisphosphonates are characterized by a pronounced selectivity in their accumulation of mineral calcium phosphate. This is based on the formation of a bidentate chelate complex of the bisphosphonates with calcium(II) ions. Bisphosphonates are preferentially adsorbed in areas of rapid bone remodeling. In bone metastases, intensive remodeling takes place compared to healthy tissue. Bisphosphonates therefore accumulate to a greater extent in bone metastases, where they initiate various processes.

On the other hand, bisphosphonates inhibit the mineralization of bone substance and bone resorption. This effect is based inter alia on inhibition of farnesyl pyrophosphate synthase (FPPS), an enzyme in the HMG-CoA reductase (mevalonate) pathway. Inhibiting the enzyme stops the production of farnesyl, an important molecule for anchoring signaling proteins to the cell membrane (FPPS), and initiates cell apoptosis. Thus, bisphosphonate derivatives as such already fulfill a therapeutic function at the cellular level.

The selective accumulation of bisphosphonates on the bone surface promotes the apoptosis of osteogenic cells, especially osteoclasts, which take up bisphosphonates to an increased extent when the bone matrix is demineralized. The increased apoptosis of osteoclasts in turn causes an antiresorptive effect.

The clinically relevant bisphosphonates are: clodronate, etidronate, pamidronate, risedronate and zoledronate.

A particularly effective radiotracer for theranostics of bone metastases has been found to be zoledronate (ZOL), a hydroxy-bisphosphonate with a heteroaromatic N unit. Zoledronate conjugated with the chelators NODAGA and DOTA (scheme 4) represents the currently most potent radio-theranostics for bone metastases.

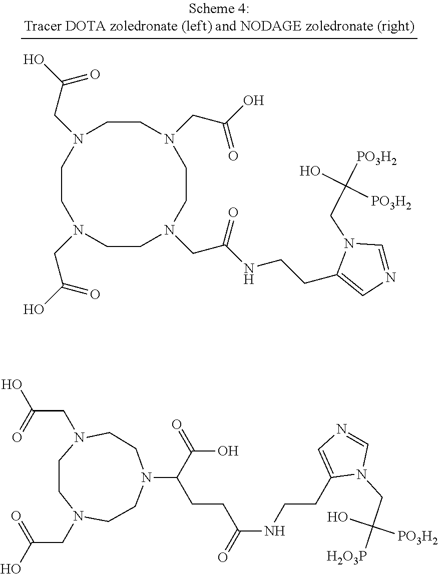

In the treatment of prostate carcinomas and metastases at an advanced stage, monomeric radiotracers with the above-described PSMA targeting vector KuE are currently used with preference. PSMA is also expressed on the surface of healthy cells. Therefore, monomeric radiotracers with PSMA targeting vector are also accumulated to a considerable extent in healthy tissue. The associated radiation dose causes various toxic side effects. These are particularly pronounced in the case of radiotracers labeled with ²²⁵Ac (²²⁵actinium), which totally and irreversibly damage the salivary glands. Therefore, the form of therapy with the ²²⁵Ac radioisotope is no longer used.

Despite a decline in the number of patients with metastatic prostate cancer in recent years as a result of improved diagnosis, the number of patients with prostate cancer metastases is still high, with about 80% of the metastases affecting bone tissue. In the case of metastatic prostate cancer, there is a severe drop in the survival rate. This is particularly true of bone metastases. In addition, bone metastases cause severe pain and considerably impair the quality of life. In general, the only remaining clinical option is palliative treatment (Gandaglia, G., et al., Impact of Metastases on Survival in Patients with Metastatic Prostate Cancer, European Urology, 2015, 68 (2), 325-334).

It is an object of the present invention to provide labeling precursors and radiotracers for a gentle and effective treatment of metastatic prostate cancer.

This object is achieved by a labeling precursor for complexing radioactive isotopes with the structure

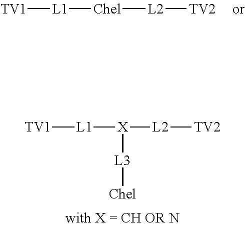

in which
a first targeting vector TV1 is selected from the group of PSMA inhibitors comprising

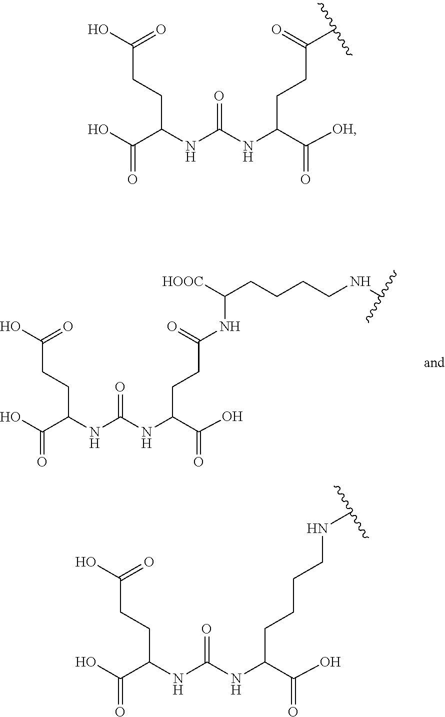 [SMILES: *C(=O)CCC(NC(=O)NC(CCC(=O)O)C(=O)O)C(=O)O ; *NCCCCC(NC(=O)CCC(NC(=O)NC(CCC(=O)O)C(=O)O)C(=O)O)C(=O)O ; *NCCCCC(NC(=O)NC(CCC(=O)O)C(=O)O)C(=O)O]

a second targeting vector TV2 is selected from the group of bisphosphonates comprising

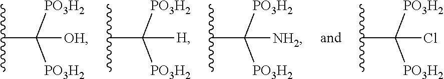

a first linker L1 has a structure selected from

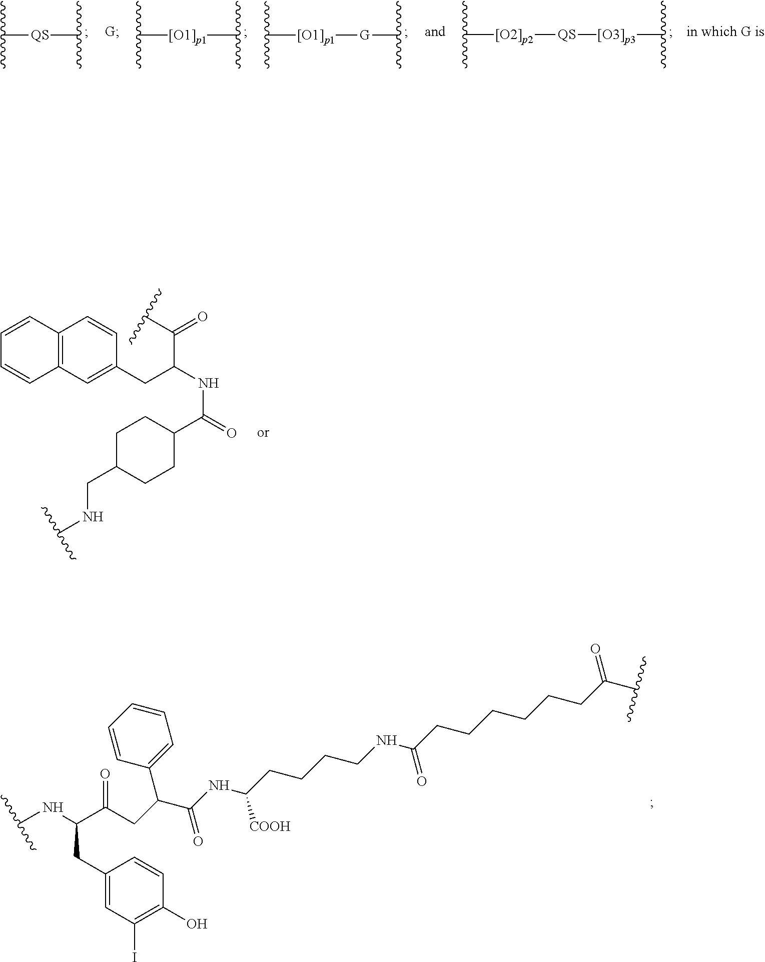

O1, O2 and O3 are independently selected from the group comprising amide radicals, carboxamide radicals, phosphinate radicals, alkyl radicals, triazole radicals, thiourea radicals, ethylene radicals, maleimide radicals, —(CH₂)—, —(CH₂CH₂O)—, —CH₂—CH(COOH)—NH— and —(CH₂)_{q}NH— with q=1, 2, 3, 4, 5, 6, 7, 8, 9 or 10;
   p1, p2 and p3 are independently selected from the set {0, 1, 2, 3, 4, 5, 6, 7, 8, 9, 10, 11, 12, 13, 14, 15, 16, 17, 18, 19, 20};
a second linker L2 has a structure selected from

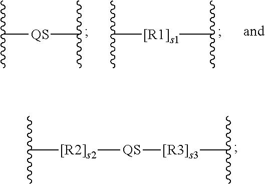

in which R1, R2 and R3 are independently selected from the group comprising amide radicals, carboxamide radicals, phosphinate radicals, alkyl radicals, triazole radicals, thiourea radicals, ethylene radicals, maleimide radicals, furan radicals, azole radicals, oxazole radicals, thiophene radicals, thiazole radicals, azine radicals, thiazine radicals, naphthalene radicals, quinoline radicals, pyrrole radicals, imidazole radicals, pyrazole radicals, tetrazole radicals, thiadiazole radicals, oxadiazole radicals, pyridine radicals, pyrimidine radicals, triazine radicals, tetrazine radicals, thiazine radicals, oxazine radicals, naphthalene radicals, chromene radicals or thiochromene radicals, —(CH₂)—, —(CH₂CH₂O)—, —CH₂—CH(COOH)—NH— and —(CH₂)_{q}NH— with q=1, 2, 3, 4, 5, 6, 7, 8, 9 or 10;
   s1, s2 and s3 are independently selected from the set {0, 1, 2, 3, 4, 5, 6, 7, 8, 9, 10, 11, 12, 13, 14, 15, 16, 17, 18 , 19, 20};
a third linker L3 has a structure selected from

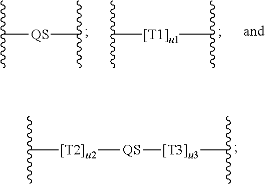

in which
   T1, T2 and T3 are independently selected from the group comprising amide radicals, carboxamide radicals, phosphinate radicals, alkyl radicals, triazole radicals, thiourea radicals, ethylene radicals, maleimide radicals, —(CH₂)—, —(CH₂CH₂O)—, —CH₂—CH(COOH)—NH— and —(CH₂)ᵥNH— with v=1, 2, 3, 4, 5, 6, 7, 8, 9 or 10;
   u1, u2 and u3 are independently selected from the set {0, 1, 2, 3, 4, 5, 6, 7, 8, 9, 10, 11, 12, 13, 14, 15, 16, 17, 18 , 19, 20};
QS is a squaric acid radical;

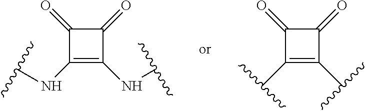 [SMILES: *Nc1c(N*)c(=O)c1=O ; *c1c(*)c(=O)c1=O]

and
a chelator Chel is selected from the group comprising H₄pypa, EDTA (ethylenediaminetetraacetate), EDTMP (diethylenetriaminepenta(methylenephosphonic acid)), DTPA (diethylenetriaminepentaacetate) and derivatives thereof, DOTA (dodeca-1,4,7,10-tetraamine tetraacetate), DOTAGA (2-(1,4,7,10-tetraazacyclododecane-4,7,10)pentanedioic acid) and other DOTA derivatives, TRITA (trideca-1,4,7,10-tetraamine tetraacetate), TETA (tetradeca-1,4,8,11-tetraamine tetraacetate) and derivatives thereof, NOTA (nona-1,4,7-triamine triacetate) and derivatives thereof, for example NOTAGA (1,4,7-triazacyclononane,1-glutaric acid,4,7-acetate), TRAP (triazacyclononanephosphinic acid), NOPO (1,4,7-triazacyclononane-1,4-bis[methylene(hydroxymethyl)phosphinic acid]-7-[methylene(2-carboxyethyl)phosphinic acid]), PEPA (pentadeca-1,4,7,10,13-pentaamine pentaacetate), HEHA (hexadeca-1,4,7,10,13,16-hexaamine hexaacetate) and derivatives thereof, HBED (hydroxybenzylethylenediamine) and derivatives thereof, DEDPA and derivatives thereof, such as H₂DEDPA (1,2-[[6-(carboxylate)pyridin-2-yl]methylamine]ethane), DFO (deferoxamine) and derivatives thereof, trishydroxypyridinone (THP) and derivatives thereof, such as YM103, TEAP (tetraazacyclodecanephosphinic acid) and derivatives thereof, AAZTA (6-amino-6-methylperhydro-1,4-diazepine N,N,N′,N′-tetraacetate) and derivatives such as DATA ((6-pentanoic acid)-6-(amino)methyl-1,4-diazepine triacetate); SarAr (1-N-(4-aminobenzyl)-3,6,10,13,16,19-hexaazabicyclo[6.6.6]eicosane-1,8-diamine) and salts thereof, (NH₂)₂SAR (1,8-diamino-3,6,10,13,16,19-hexaazabicyclo[6.6.6]eicosane) and salts and derivatives thereof, aminothiols and derivatives thereof.

Appropriate embodiments of the labeling precursor of the invention are characterized by the following features in any combination, insofar as the features are not mutually exclusive, and according to which:
the chelator Chel is DOTA;
the chelator Chel is H₄pypa;
the chelator Chel is DATA;
the chelator Chel is DOTAGA;
the second linker L2 comprises at least one radical selected from

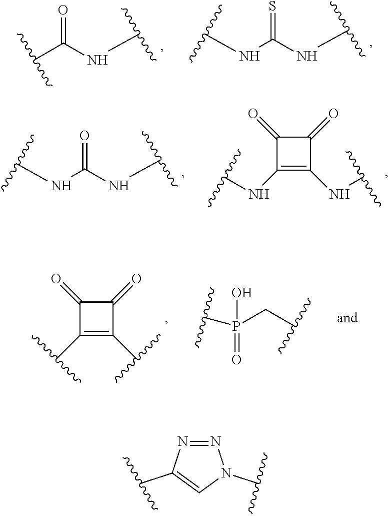

the second linker L2 includes at least one squaric acid radical

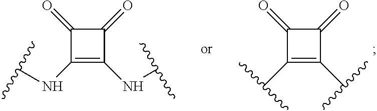 [SMILES: *Nc1c(N*)c(=O)c1=O ; *c1c(*)c(=O)c1=O]

the second linker L2 comprises at least one radical selected from

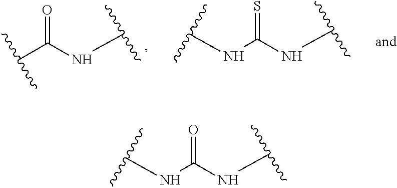 [SMILES: *NC(*)=O ; *NC(=S)N* ; *NC(=O)N*]

the linkers L1 and L2 are the same (L1=L2);
the linkers L1 and L3 are the same (L1=L2);
the linkers L2 and L3 are the same (L2=L3);
the linkers L1, L2 and L3 are the same (L1=L2=L3);
the second linker L2 comprises a radical selected from the group comprising a radical of

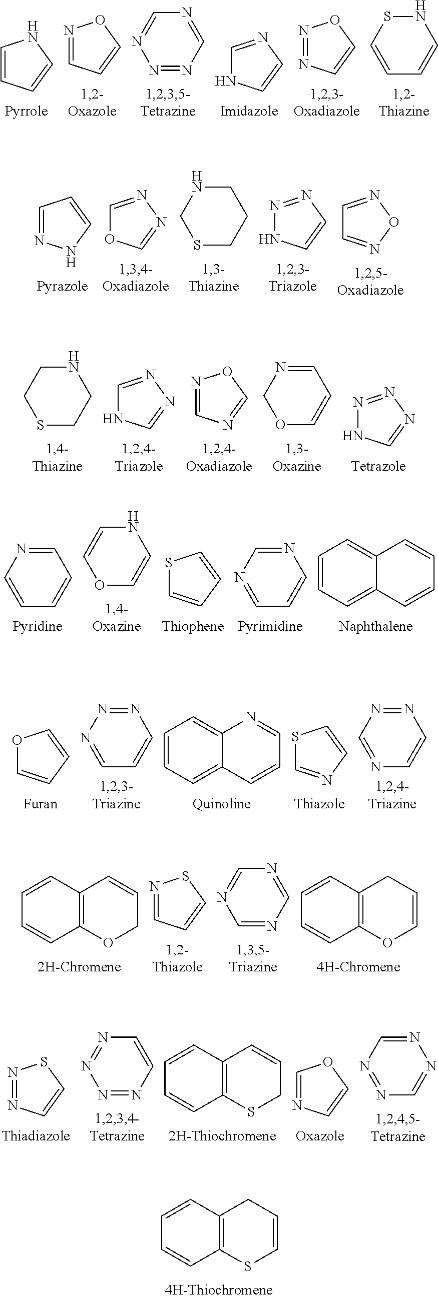

and derivatives of the above radicals;
the second linker L2 includes at least one imidazole radical

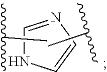

G is

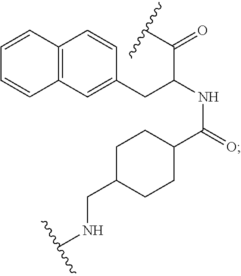 [SMILES: *NCC1CCC(C(=O)NC(Cc2ccc3ccccc3c2)C(*)=O)CC1]

the labeling precursor has the structure

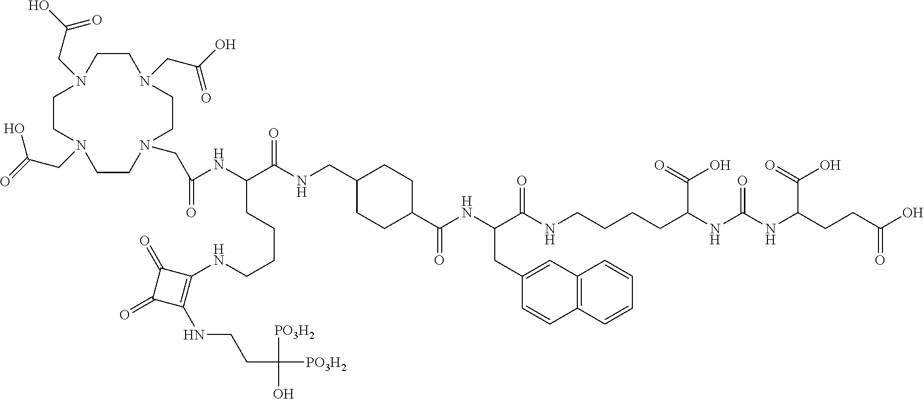

The invention is elucidated in detail hereinafter with reference to figures and examples. The figures show:

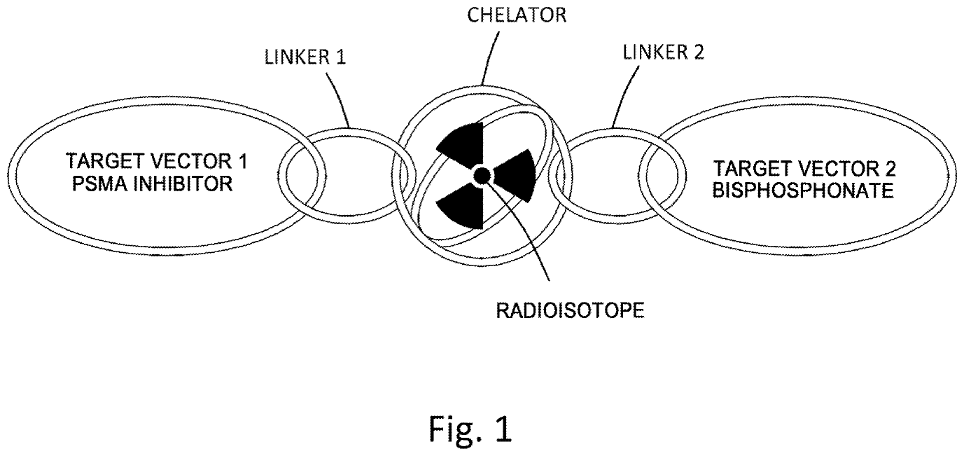

FIG. 1 . . . the functional components of a radiotracer;

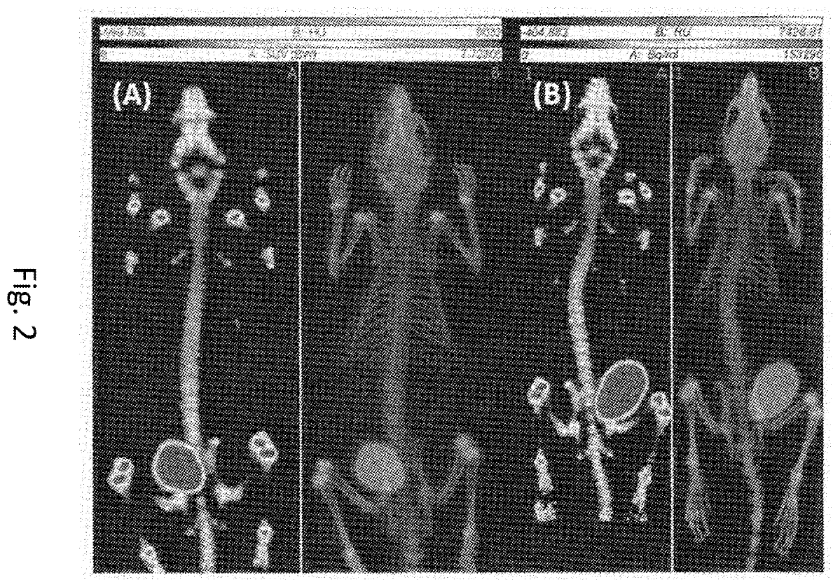

FIG. 2 . . . in vivo PET images of Wistar rats;

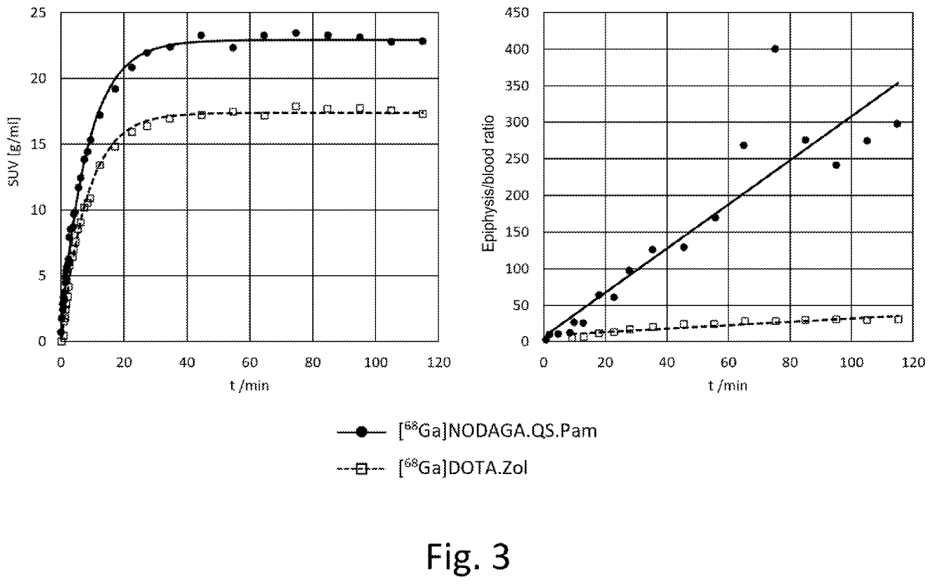

FIG. 3 . . . diagrams of the progression of SUV and the PET signal ratio of epiphysis and blood against time; and

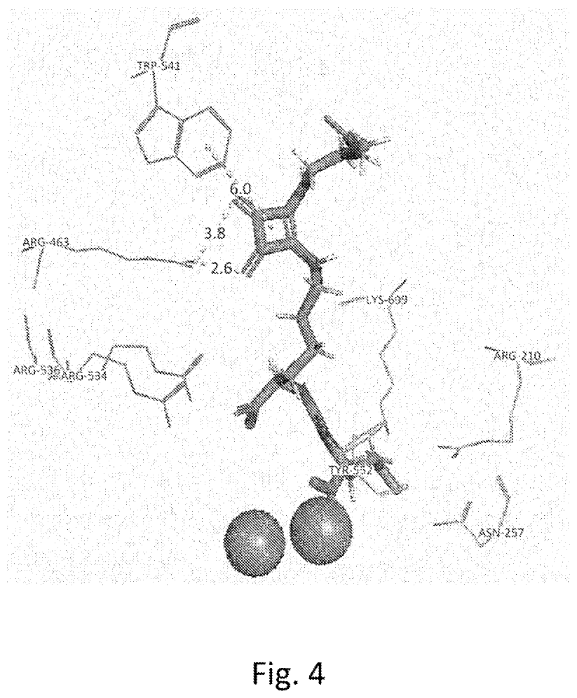

FIG. 4 . . . the result of a docking simulation for the PSMA binding pocket;

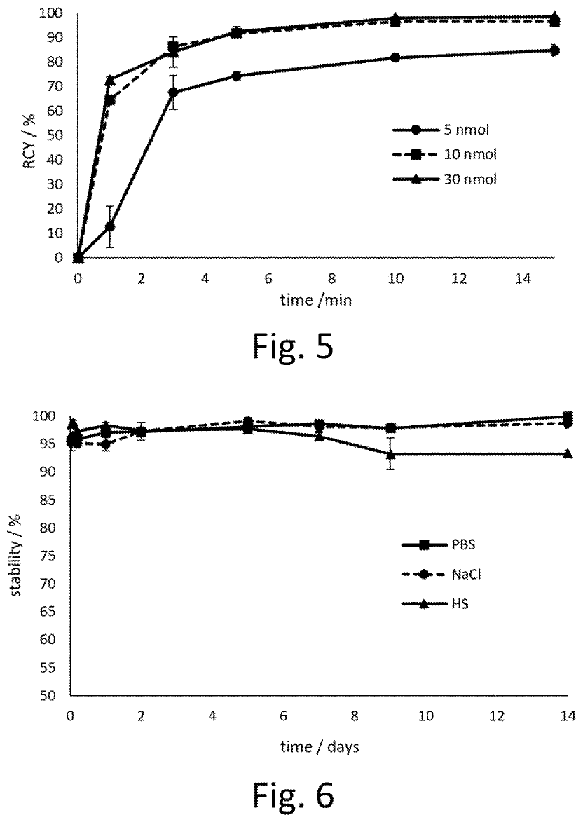

FIG. 5 . . . the labeling kinetics of a labeling precursor;

FIG. 6 . . . the stability of a radiotracer in physiological medium;

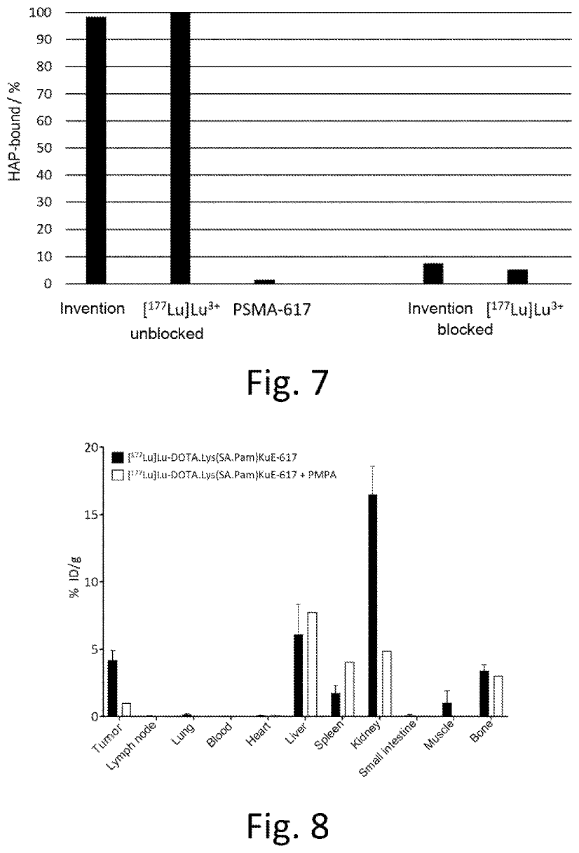

FIG. 7 . . . binding affinities for hydroxyapatite;

FIG. 8 . . . ex vivo organ accumulations with and without blocking of PSMA.

### EXAMPLE OF AMIDE COUPLING

In the invention, the chelator Chel, the targeting vectors TV1, TV2, and the linkers L1, L2 are preferably conjugated by an amide coupling reaction. Amide coupling, which forms the backbone of proteins, is the most commonly used reaction in medicinal chemistry. A generic example of an amide coupling is shown in scheme 5.

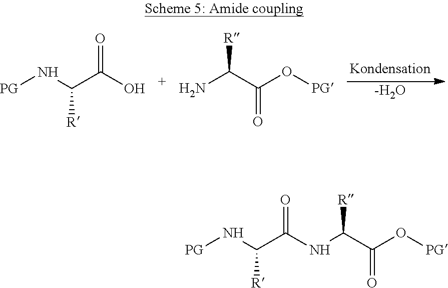

Owing to a virtually unlimited set of readily available carboxylic acid and amine derivatives, amide coupling strategies open up a simple route to the synthesis of novel compounds. Numerous reagents and protocols for amide coupling are known to those skilled in the art. The most common amide coupling strategy is based on the condensation of a carboxylic acid with an amine. The carboxylic acid is generally activated for this purpose. Remaining functional groups are protected prior to activation. The reaction is effected in two steps, either in one reaction medium (single pot) with direct conversion of the activated carboxylic acid or in two steps with isolation of an activated “trapped” carboxylic acid and reaction with an amine.

The carboxylate reacts here with a coupling agent to form a reactive intermediate that can be isolated or reacted directly with an amine. Numerous reagents are available for carboxylic acid activation, such as acid halides (chloride, fluoride), azides, anhydrides, or carbodiimides. In addition, esters such as pentafluorophenyl or hydroxysuccinic imido esters can be formed as reactive intermediates. Intermediates derived from acyl chlorides or azides are highly reactive. However, harsh reaction conditions and high reactivity are often a barrier to use for sensitive substrates or amino acids. In contrast, amide coupling strategies that use carbodiimides such as DCC (dicyclohexylcarbodiimide) or DIC (diisopropylcarbodiimide) open up a wide range of applications. Commonly, especially in solid-phase synthesis, additives are used to improve reaction efficiency. Aminium salts are highly efficient peptide coupling reagents with short reaction times and minimal racemization. With some additives, for example HOBt, racemization can even be completely avoided. Aminium reagents are used in equimolar amounts relative to the carboxylic acid in order to prevent excessive reaction with the free amine of the peptide. Phosphonium salts react with carboxylate, which generally requires two equivalents of a base, for example DIEA. A key advantage of phosphonium salts over iminium reagents is that phosphonium does not react with the free amino group of the amine component. This enables couplings in equimolar ratios of acid and amine and helps to avoid intramolecular cyclization of linear peptides and excessive use of costly amine components.

A comprehensive summary of reaction strategies and reagents for amide coupling can be found in the review articles:
Analysis of Past and Present Synthetic Methodologies on Medicinal Chemistry: Where Have All the New Reactions Gone?; D. G. Brown, J. Boström; J. Med. Chem. 2016, 59 (10), 4443-4458;
Peptide Coupling Reagents, More than a Letter Soup; A. El-Faham, F. Albericio; Chem. Rev. 2011, 111 (11), 6557-6602;
Rethinking amide bond synthesis; V. R. Pattabiraman, J. W. Bode; Nature, Vol. 480 (2011) 471-479;
Amide bond formation: beyond the myth of coupling reagents; E. Valeur, M. Bradley; Chem. Soc. Rev., 2009, 38, 606-631.

Many of the chelators used in accordance with the invention, such as DOTA in particular, have one or more carboxy or amide groups. Accordingly, these chelators can be readily conjugated to the linkers L1, L2 using any of the amide coupling strategies known in the art. Schemes 6 and 7 show examples of coupling of the linker targeting vector unit L1-TV1 with a chelator Chel; schemes 8-10 show examples of coupling of L2-TV2 with a chelator Chel.

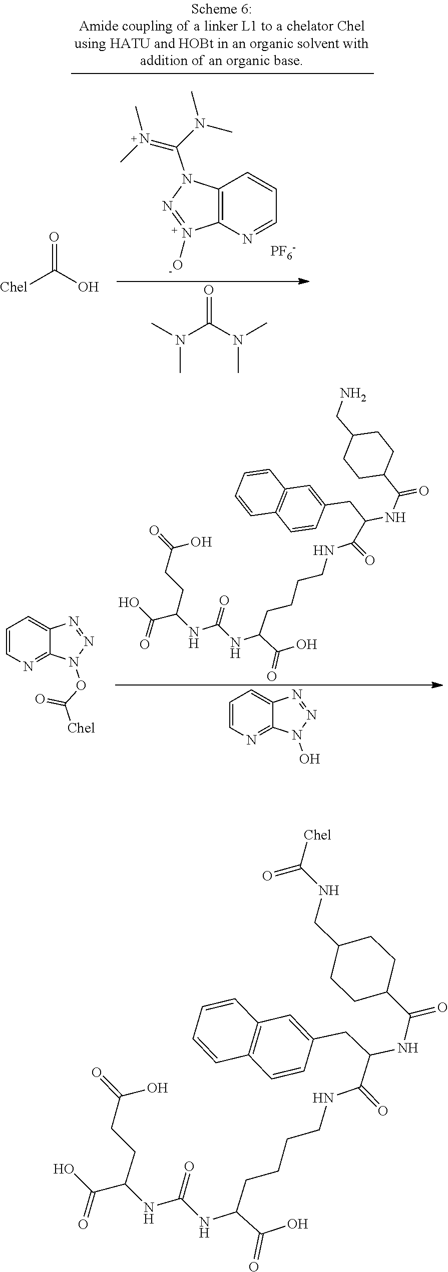

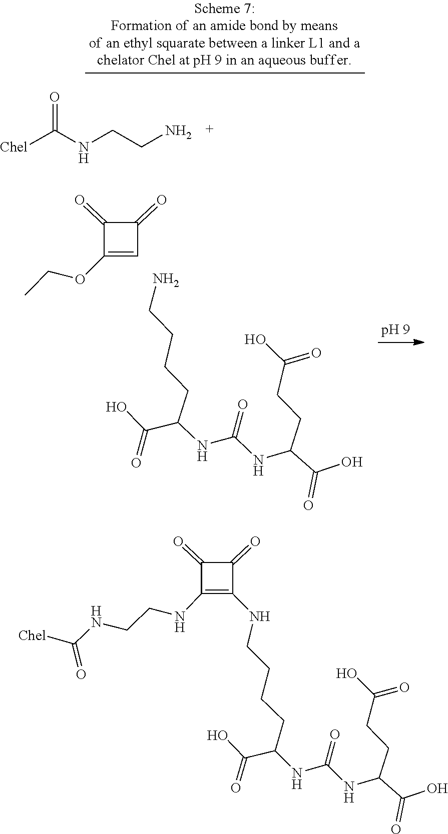

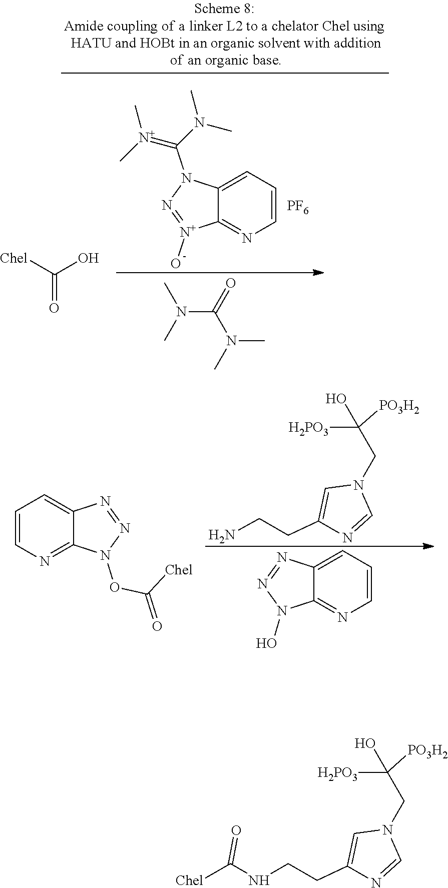

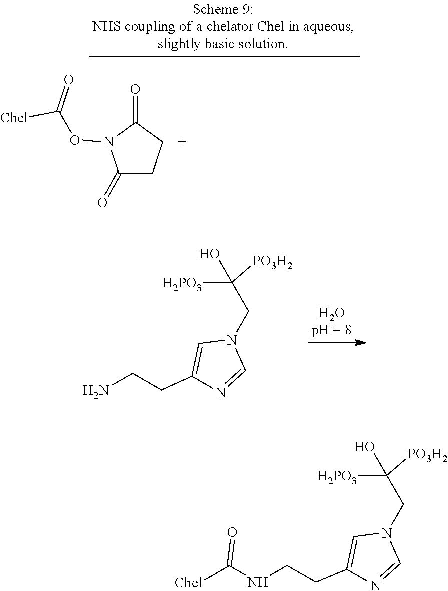

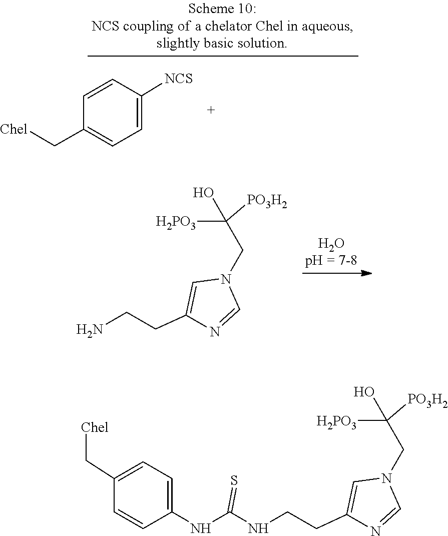

### Chelator Chel for Radioisotope Labeling

The chelator Chel is intended for labeling of the labeling precursor of the invention with a radioisotope selected from the group consisting of ⁴⁴Sc, ⁴⁷Sc, ⁵⁵Co, ⁶²Cu, ⁶⁴Cu, ⁶⁷Cu, ⁶⁶Ga, ⁶⁷Ga, ⁶⁸Ga, ⁸⁹Zr, ⁸⁶Y, ⁹⁰Y, ⁸⁹Zr, ⁹⁰Nb, ^{90m}Tc, ¹¹¹In, ¹³⁵Sm, ¹⁴⁰Pr, ¹⁵⁹Gd, ¹⁴⁹Tb, ¹⁶⁰Tb, ¹⁶¹Tb, ¹⁶⁵Er, ¹⁶⁶Dy, ¹⁶⁶Ho, ¹⁷⁵Yb, ¹⁷⁷Lu, ¹⁸⁶Re, ¹⁸⁸Re, ²¹¹At, ²¹²Pb, ²¹³Bi, ²²⁵Ac and ²³²Th. A variety of chelating agents for complexing the above radioisotopes are known in the art. Scheme 11 shows examples of chelators used in accordance with the invention.

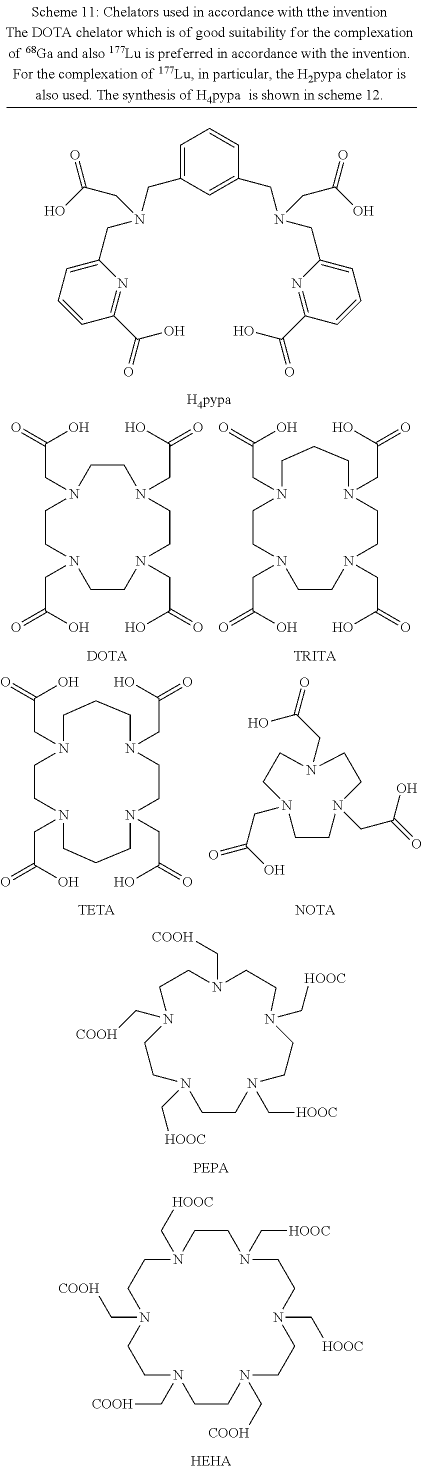

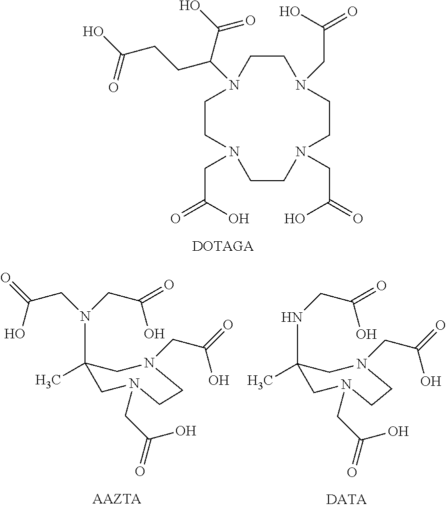 [SMILES: O=C(O)CCC(C(=O)O)N1CCN(CC(=O)O)CCN(CC(=O)O)CCN(CC(=O)O)CC1 ; CC1(N(CC(=O)O)CC(=O)O)CN(CC(=O)O)CCN(CC(=O)O)C1 ; CC1(NCC(=O)O)CN(CC(=O)O)CCN(CC(=O)O)C1 ; "DATA"]

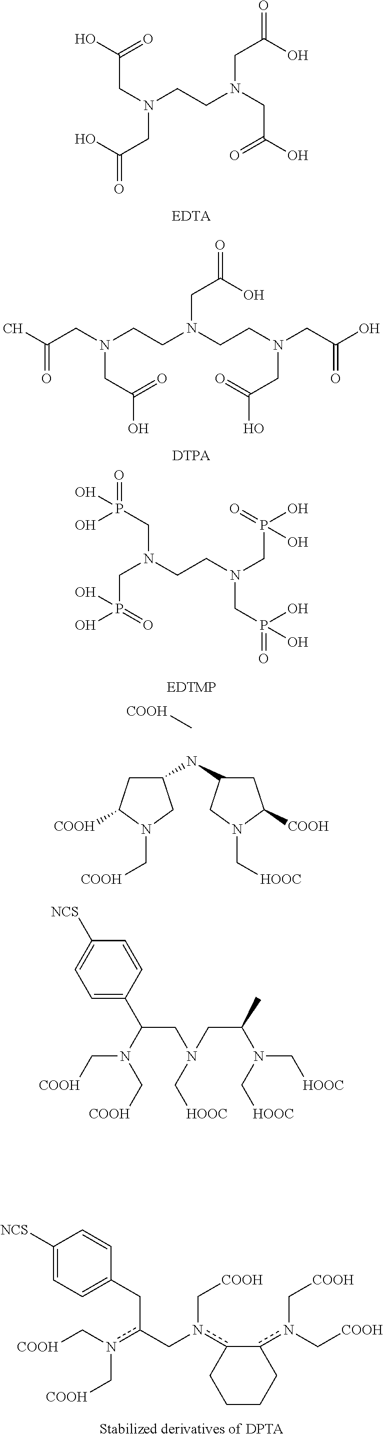

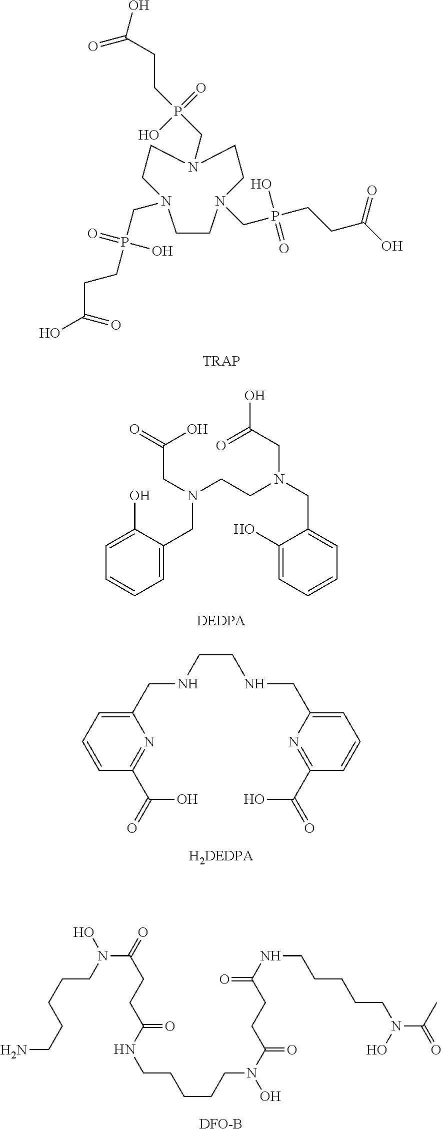

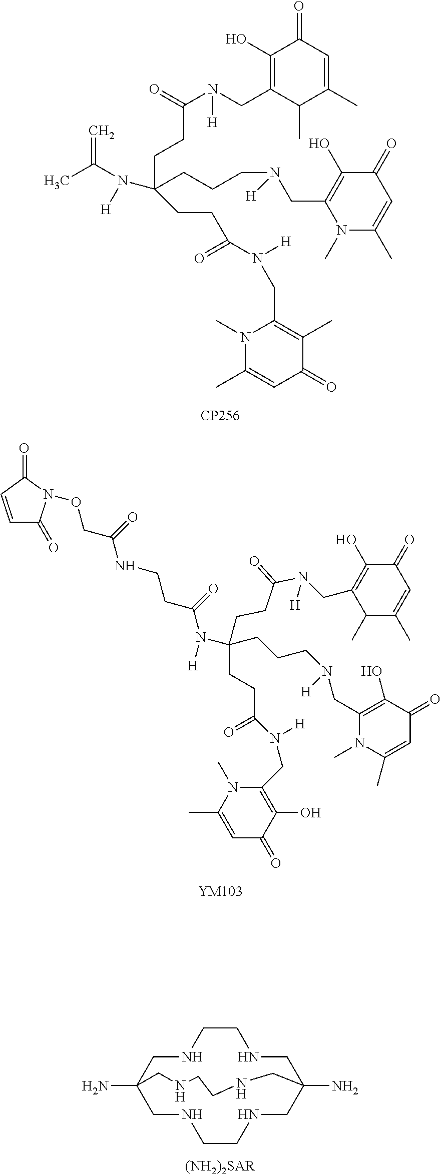

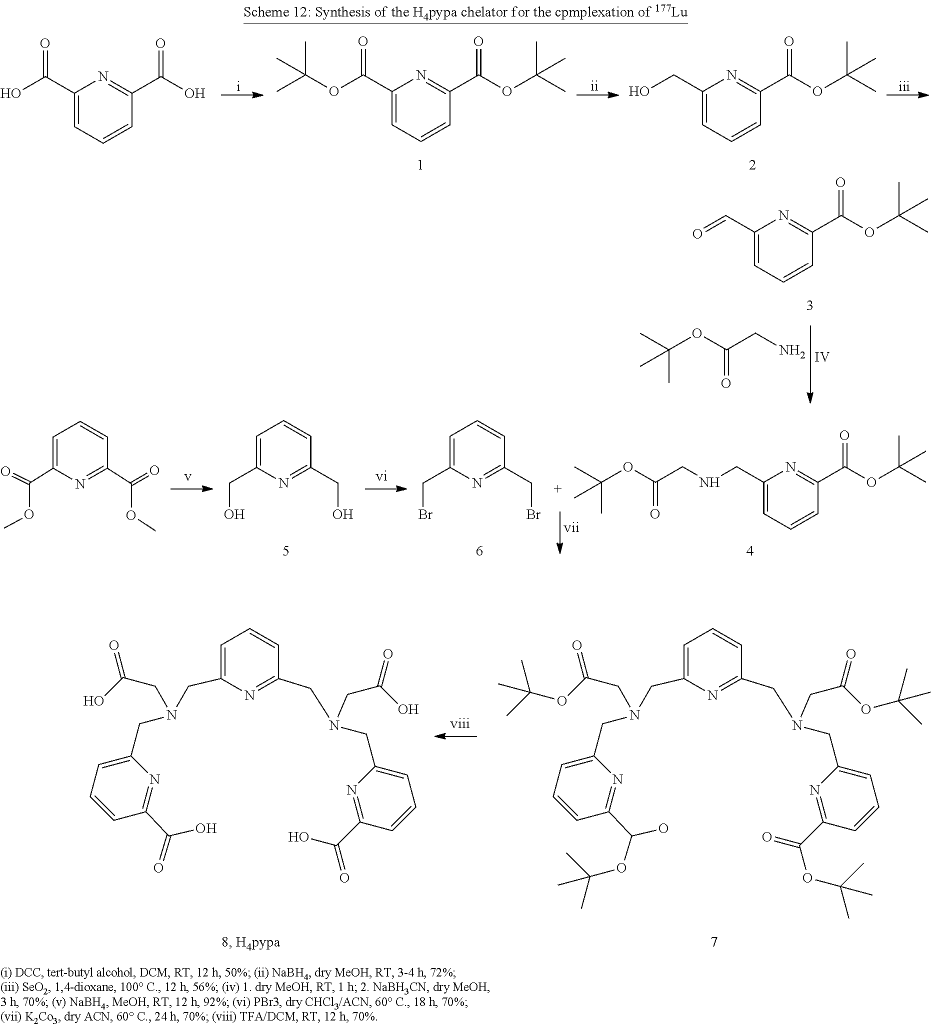

### Radioisotopes

For nuclear-medical theranostics (diagnosis and therapy), in particular, the radioisotopes ⁶⁸Ga or ¹⁷⁷Lu used. The invention also provides for the use of radioisotopes selected from the group comprising ⁴⁴Sc, ⁴⁷Sc, ⁵⁵Co, ⁶²Cu, ⁶⁴Cu, ⁶⁷Cu, ⁶⁶Ga, ⁶⁷Ga, ⁶⁸Ga, ⁸⁹Zr, ⁹⁰Nb, ^{90m}Tc, ¹¹¹In, ¹³⁵Sm, ¹⁴⁰Pr, ¹⁵⁹Gd, ¹⁴⁹Tb, ¹⁶⁰Tb, ¹⁶¹Tb, ¹⁶⁵Er, ¹⁶⁶Dy, ¹⁶⁶Ho, ¹⁷⁵Yb, ¹⁷⁷Lu, ¹⁸⁶Re, ¹⁸⁸Re, ²¹¹At, ²¹²Pb, ²¹³Bi, ²²⁵Ac and ²³²Th.

Structural formulae of labeling precursors of the invention are listed below:

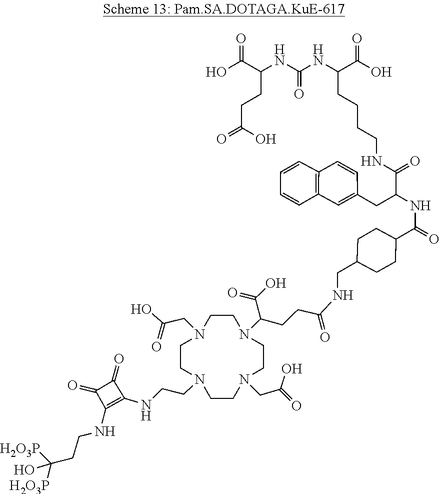

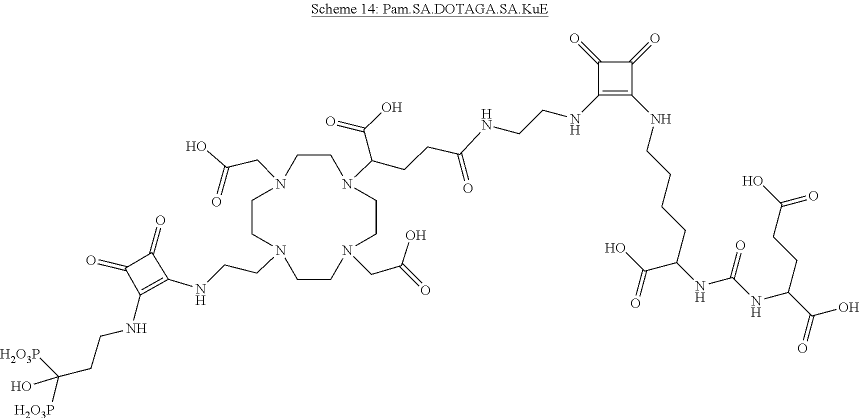

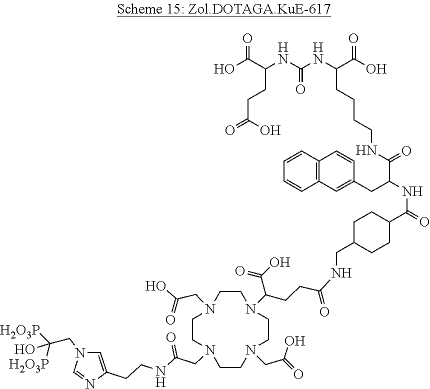

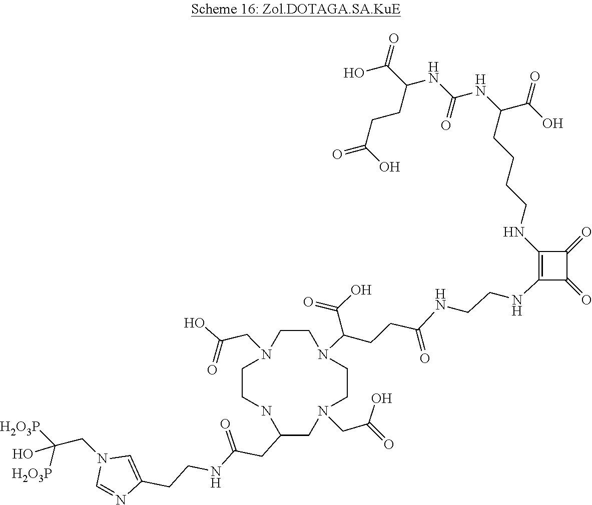

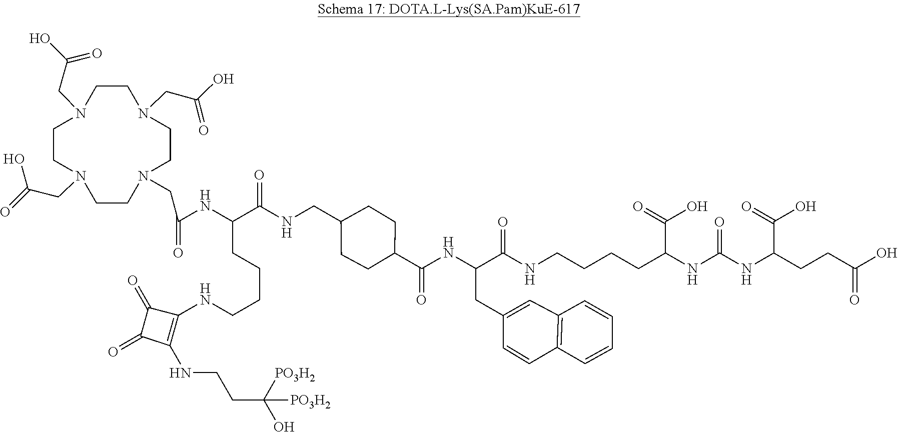

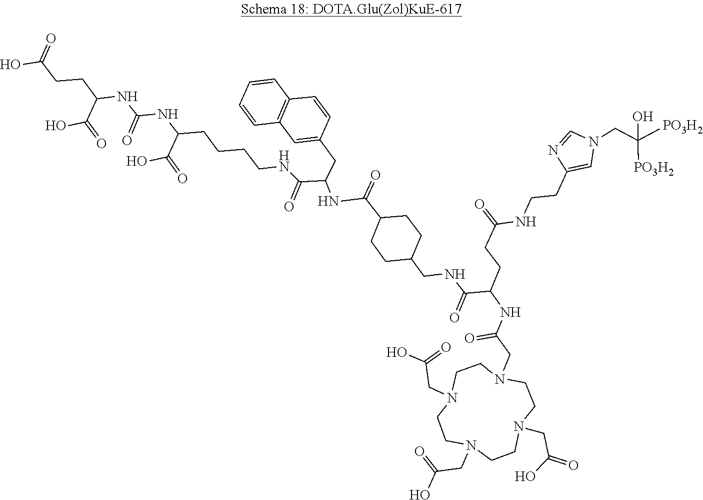

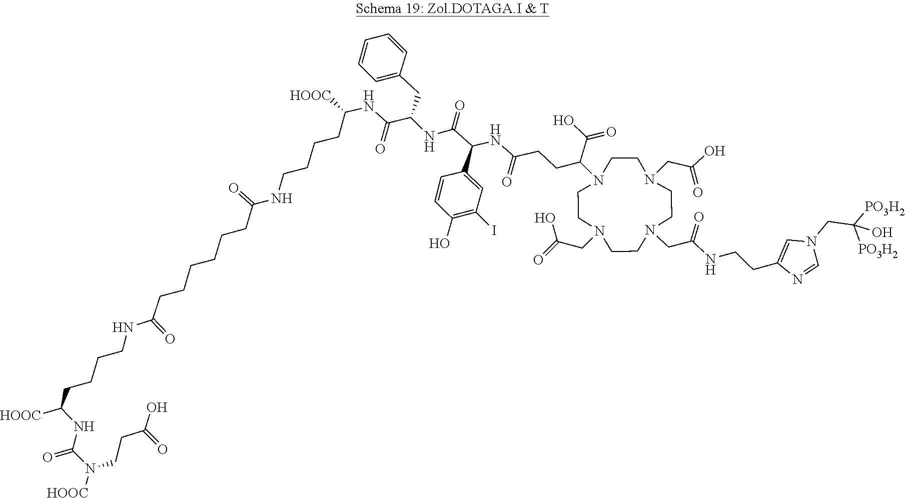

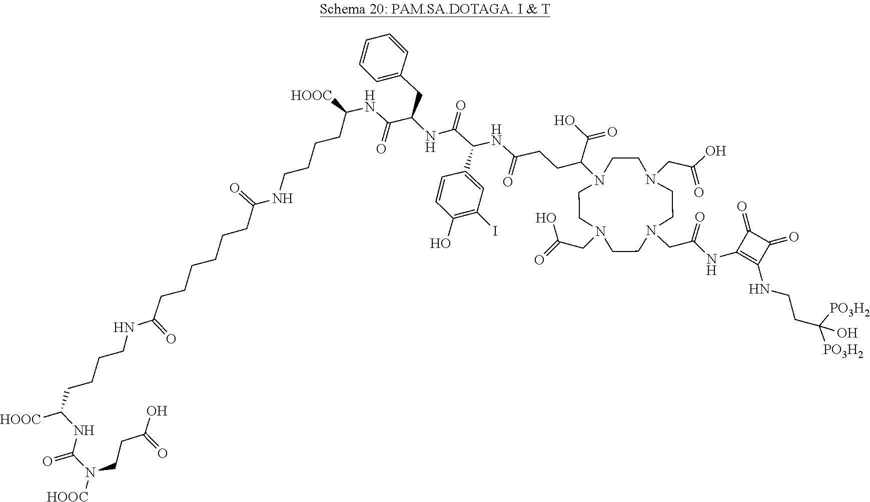

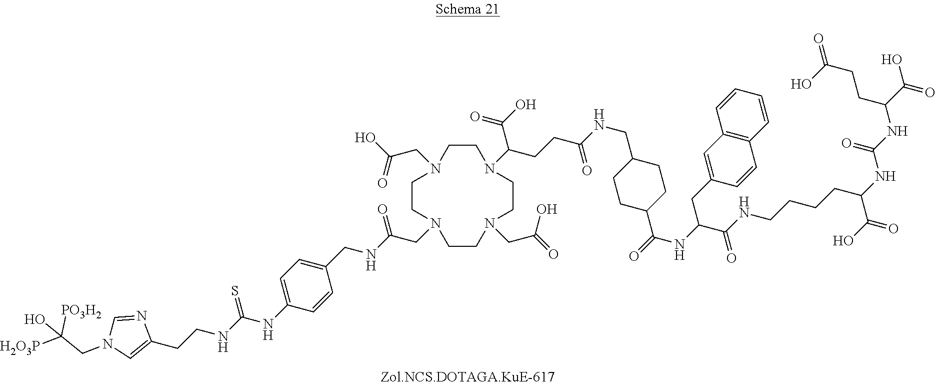

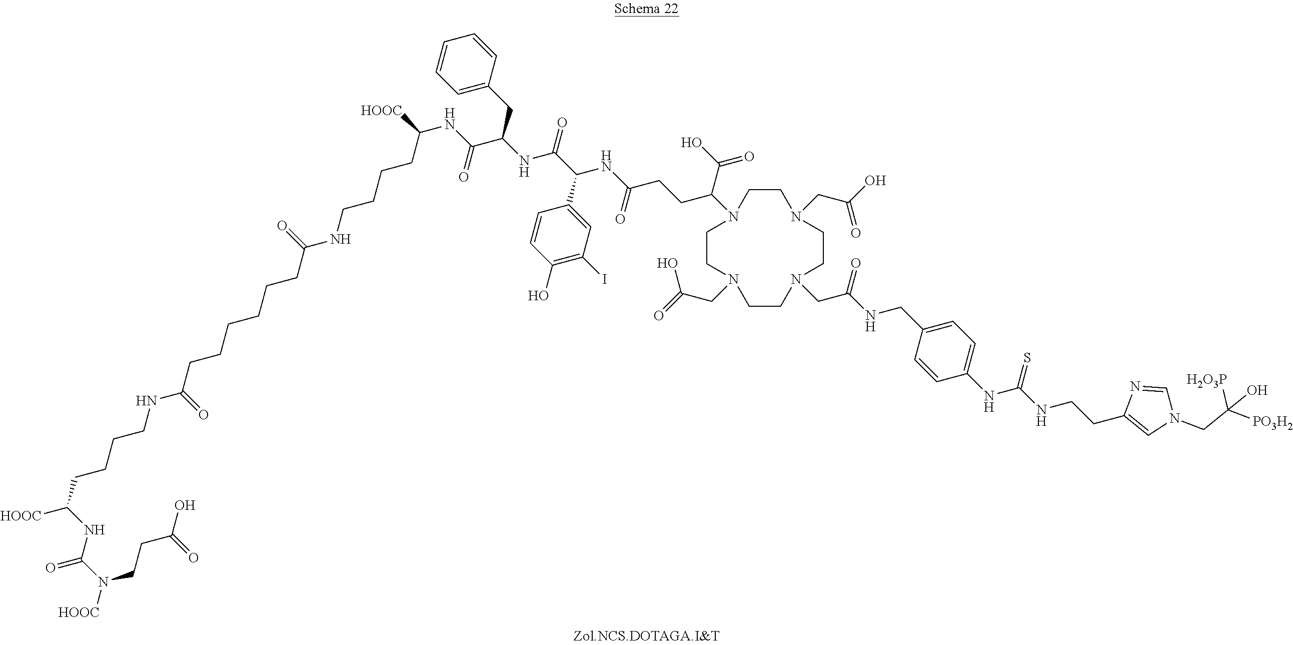

### Example 1: Squaric Acid as Affinity Promoter for Bisphosphonates

The inventors have found that, surprisingly, squaric acid as a component of a linker of a bisphosphonate targeting vector increases affinity for hydroxyapatite in bone tissue. This beneficial effect is demonstrated by the adsorption therms of conjugates of the chelator NODAGA with squaric acid pamidronate (NODAGA.QS.Pam) and NODAGA with zoledronate (NODAGA.Zol). For this purpose, the adsorption therms are determined by the method of Langmuir and Freundlich.

For comparison, schemes 23 and 24 show conjugates of the chelator NODAGA with squaric acid pamidronate (NODAGA.QS.Pam) and NODAGA with zoledronate (NODAGA.Zol), and also the respective coefficients of adsorption K_{LF}, measured by the method of Langmuir and Freundlich.

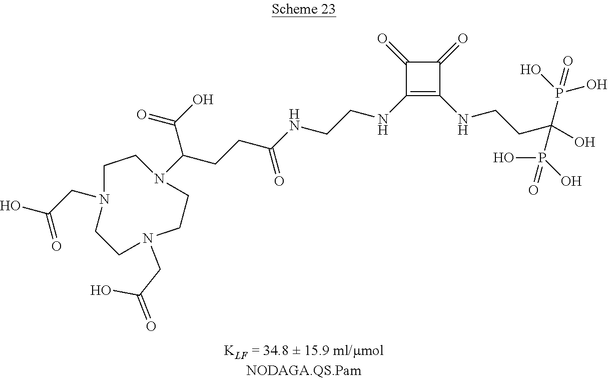 [SMILES: O=C(O)CN1CCN(CC(=O)O)CCN(C(CCC(=O)NCCNc2c(NCCC(O)(P(=O)(O)O)P(=O)(O)O)c(=O)c2=O)C(=O)O)CC1]

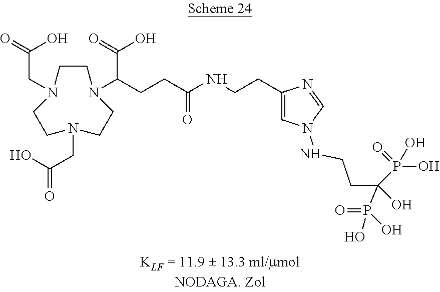 [SMILES: O=C(O)CN1CCN(CC(=O)O)CCN(C(CCC(=O)NCCc2cn(NCCC(O)(P(=O)(O)O)P(=O)(O)O)cn2)C(=O)O)CC1]

The coefficient of adsorption KLF of the conjugate NODAGA.QS.Pam is about three times greater than that of NODAGA.Zol, which contains an imidazole radical instead of a squaric acid group. It is immediately apparent from this that squaric acid significantly increases the affinity of the bisphosphonate group for bone tissue.

Moreover, in vivo PET (positron emission tomography) studies on young, healthy Wistar rats using the radiotracer [⁶⁸Ga]Ga-NODAGA.QS.Pam (cf. FIG. 2) show high accumulation in the epiphyses, which in young animals—analogously to bone metastases—are characterized by rapid renewal and remodeling of bone tissue.

Compared to published SUVs (Standardized Uptake Value, https://de.wikipedia.org/wiki/SUV_(Nuklearmedizin)) for the PET radiolabel [⁶⁸Ga]Ga-DOTA.Zol with SUV_{epiphysis}=17.4, it is possible with [⁶⁸Ga]NODAGA.QS.Pam to achieve a noticeably increased value of SUV_{epiphysis}=22.9 (cf. FIG. 3).

In addition, renal excretion of [⁶⁸Ga]NODAGA.QS.Pam (% IDᵣₑₙₐₗ=40+4.60 min p.i.) is faster than for [⁶⁸Ga]Ga-DOTA.Zol (% IDᵣₑₙₐₗ=33+17, 60 min p.i.). For [⁶⁸Ga]Ga-NODAGA.QS.Pam—as shown in FIG. 3—this results in a higher epiphysis to blood ratio of 299.1, compared to 30.3 for [⁶⁸Ga]Ga-DOTA.Zol, and correspondingly better PET image contrast (or signal-to-noise ratio).

### Example 2: Synthesis of the KuE Unit

Synthesized as targeting vector for PSMA is, for example, the PSMA inhibitor L-lysine-urea-L-glutamate (KuE) using a known method according to Benešová et al. (Linker Modification Strategies To Control the Prostate-Specific Membrane Antigen (PSMA)-Targeting and Pharmacokinetic Properties of DOTA-Conjugated PSMA Inhibitors; J Med Chem, 2016, 59 (5), 1761-1775) (cf. scheme 25). Lysine bound to a solid phase, especially to a polymer resin, and protected with tert-butyloxycarbonyl (tert-butyl) is reacted here with doubly tert-butyl-protected glutamic acid. After activation of the protected glutamic acid by triphosgene and coupling to the lysine bound to the solid phase, L-lysine-urea-L-glutamate (KuE) is eliminated using TFA and at the same time completely deprotected. The product can then be separated from free lysine by semipreparative HPLC with a yield of 71%.

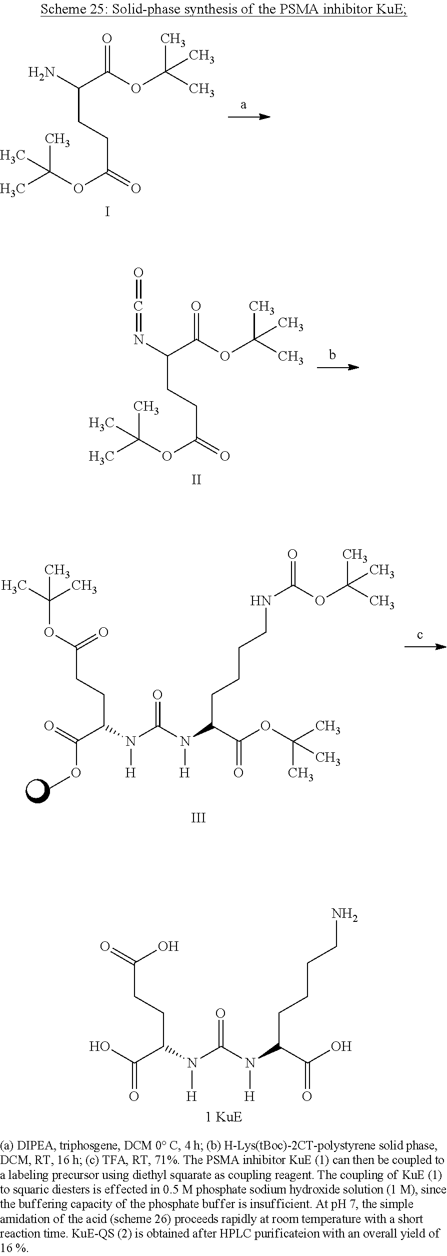

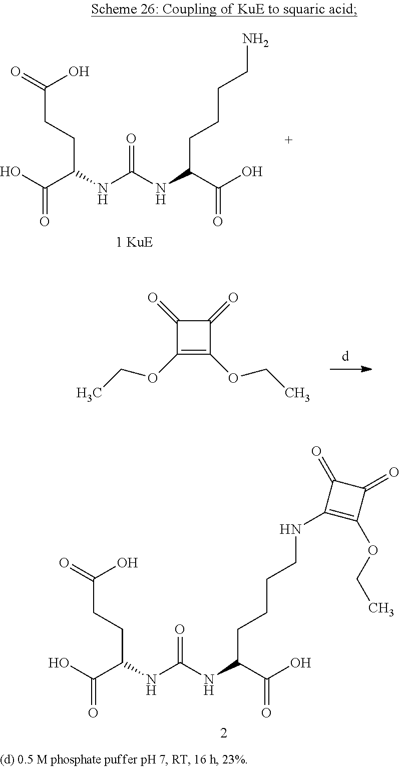

The KuE squaric acid monoester obtained in this way is storable and can be used as a building block for further syntheses.

### Example 3: Solid Phase-Based Synthesis of the Kue Unit and of the PSMA-617 Linker

The glutamate-urea-lysine binding motif KuE is conjugated with an aromatic linker unit by a method developed by Benešová et al. (Linker Modification Strategies To Control the Prostate-Specific Membrane Antigen (PSMA)-Targeting and Pharmacokinetic Properties of DOTA-Conjugated PSMA Inhibitors; J Med Chem, 2016, 59 (5), 1761-1775). The synthesis reported by Benešová et al. was modified slightly (cf. scheme 27).

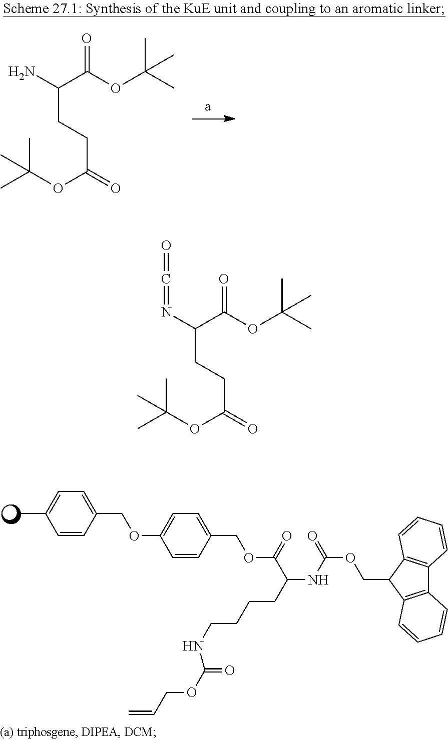 [SMILES: CC(C)(C)OC(=O)CCC(N)C(=O)OC(C)(C)C ; CC(C)(C)OC(=O)CCC(N=C=O)C(=O)OC(C)(C)C ; C=CCOC(=O)NCCCCC(NC(=O)OCC1c2ccccc2-c2ccccc21)C(=O)OCc1ccc(OCc2ccc(C)cc2)cc1]

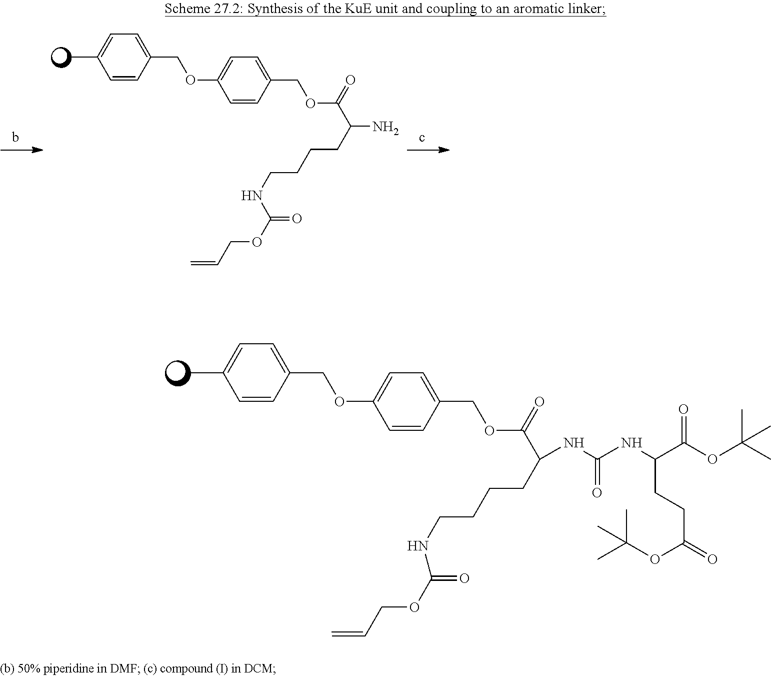 [SMILES: C=CCOC(=O)NCCCCC(N)C(=O)OCc1ccc(OCc2ccc(C)cc2)cc1 ; C=CCOC(=O)NCCCCC(NC(=O)NC(CCC(=O)OC(C)(C)C)C(=O)OC(C)(C)C)C(=O)OCc1ccc(OCc2ccc(C)cc2)cc1]

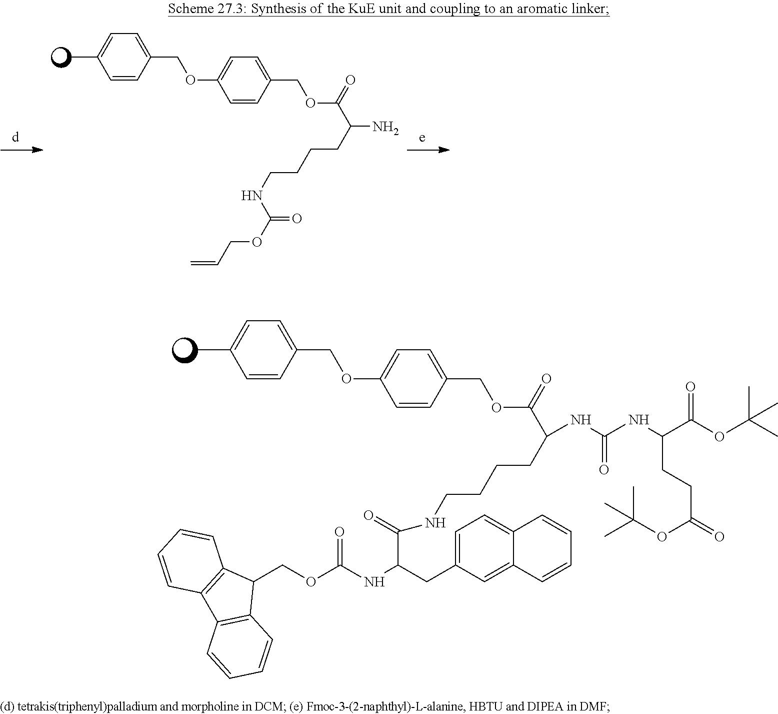 [SMILES: C=CCOC(=O)NCCCCC(N)C(=O)OCc1ccc(OCc2ccc(C)cc2)cc1 ; Cc1ccc(COc2ccc(COC(=O)C(CCCCNC(=O)C(Cc3ccc4ccccc4c3)NC(=O)OCC3c4ccccc4-c4ccccc43)NC(=O)NC(CCC(=O)OC(C)(C)C)C(=O)OC(C)(C)C)cc2)cc1]

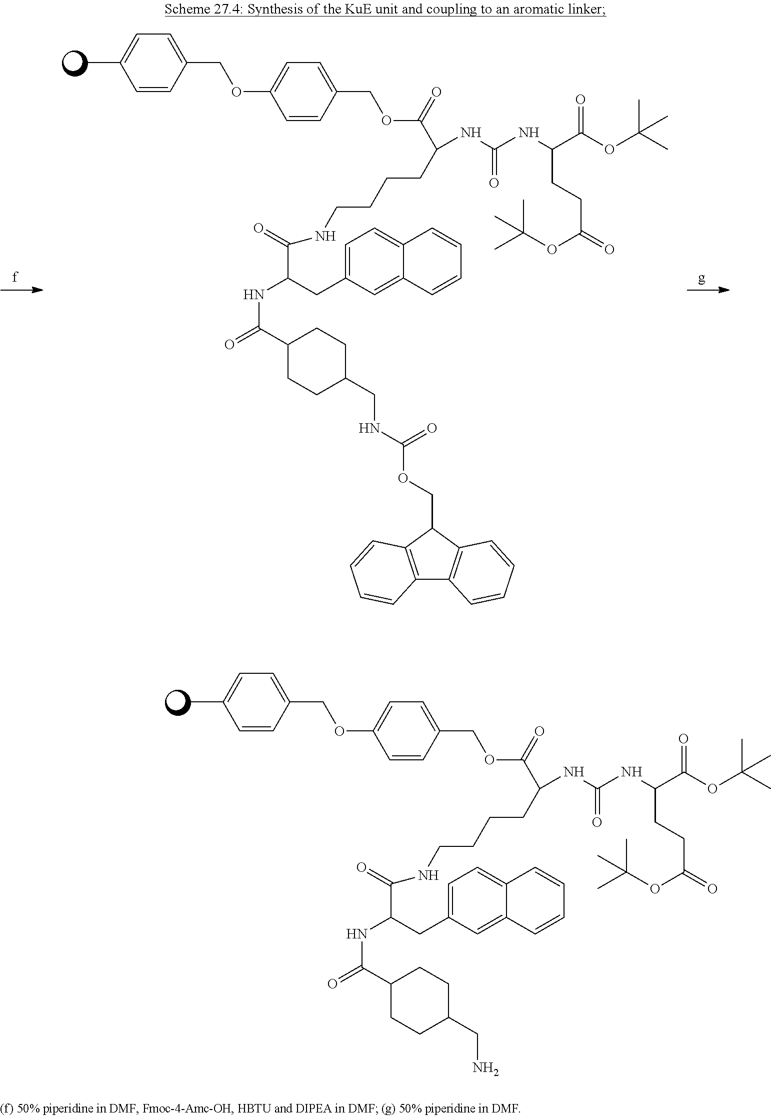

### Example 4: Synthesis of the Labeling Precursor Pam.QS.DOTAGA.KuE-617

First, the DOTAGA substructure is synthesized. This was done with a yield of 74%.

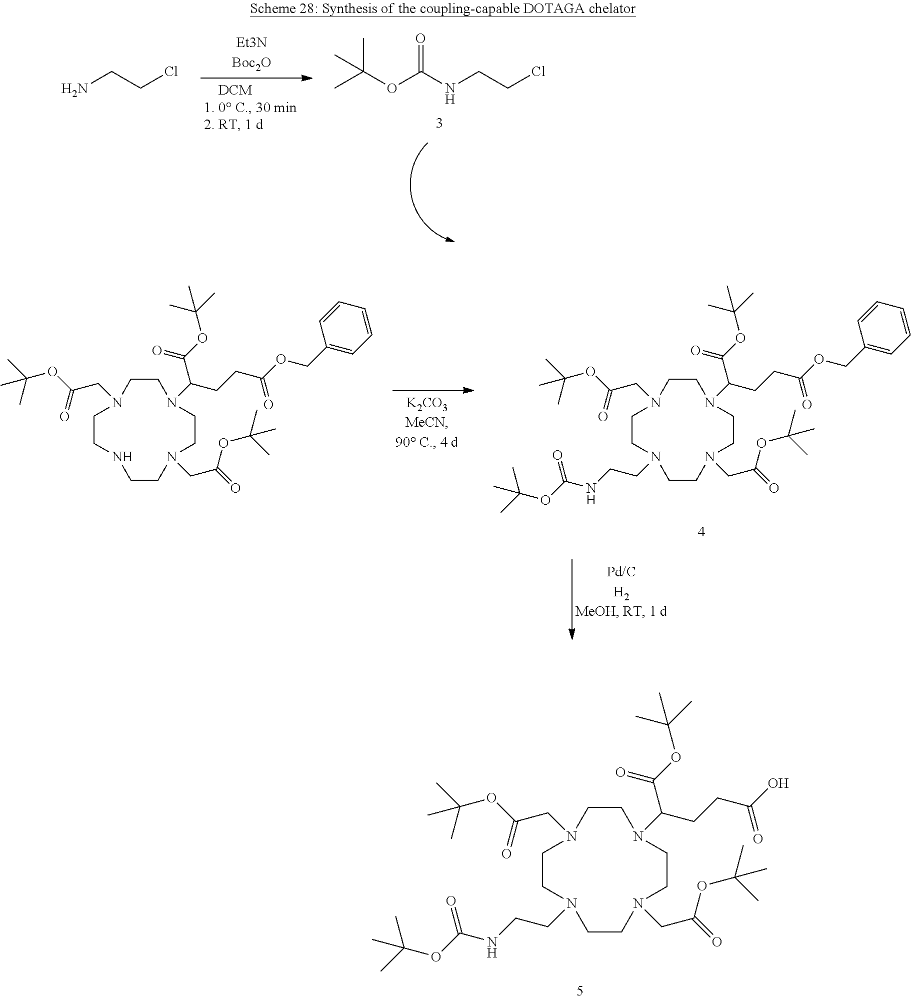

The synthesis proceeds from the commercially available DO2A(tBu)-GABz, functionalized on the secondary amine with a Boc-protected amino group.

The benzyl protecting group of the glutaric acid side chain of DOTAGA(COOtBu)₃(NHBoc)-GABz (4) is reductively removed to allow coupling to the targeting vector.

The PSMA-617 linker is then coupled to the chelator (5) via amide coupling.

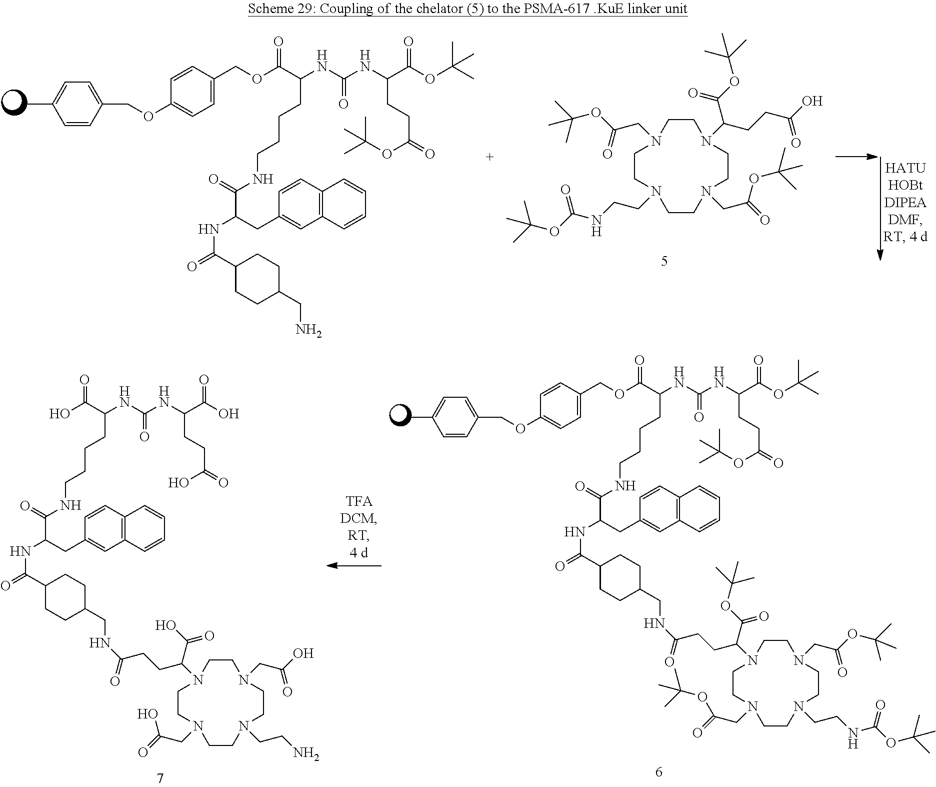

The coupling of the chelator (5) to the KuE-bound linker is described in scheme 29. The protected PSMA-617 derivative (6) obtained by the amide coupling is deprotected using trifluoroacetic acid (TFA) and detached from the solid phase. The overall yield of the two-stage synthesis was 6% after HPLC purification.

In the last step, the pamidronate-squaric acid unit is synthesized and coupled to compound (7) (scheme 30). Proceeding from B-alanine, pamidronate (8) is first prepared and coupled to squaric diester in aqueous phosphate buffer with a pH of 7. The conjugation of the pamidronate-squaric acid group (9) with DOTAGA.KuE-617 (7) is effected in aqueous phosphate buffer with a pH of 9 (cf. scheme 30). The inventive labeling precursor Pam.QS.DOTAGA.KuE-617 (10) is obtained with a yield of 49% after HPLC purification.

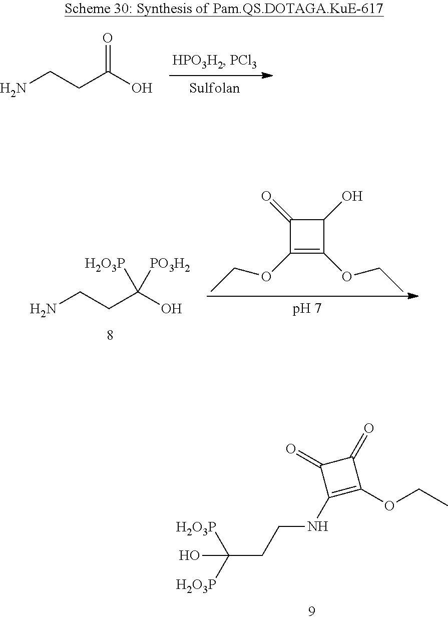

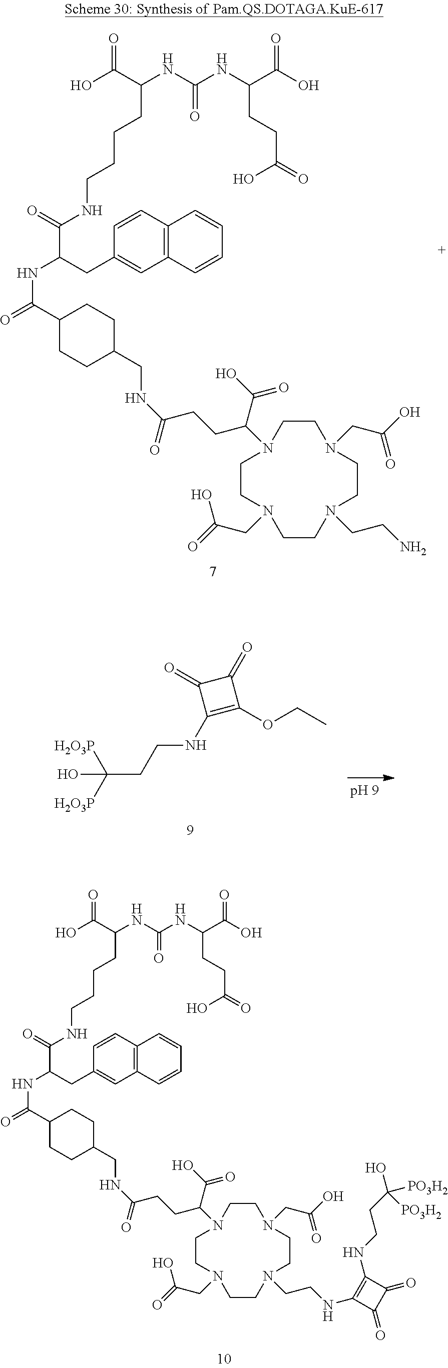

### Example 5: Synthesis of the Labeling Precursor DOTA.L-Lys(SA.Pam)KuE-617

The synthesis of the labeling precursor DOTA.L-Lys(SA.Pam)KuE-617 shown in scheme 17 is represented in scheme 31. First, the first targeting vector KuE bound to the solid phase and the aromatic linker conjugated thereto (structure (11) in scheme 31) are synthesized in the same way as shown in scheme 27. Then orthogonally protected lysine is conjugated to the linker as bridging unit X. Fmoc deprotection (structure (12)) is followed by coupling with DOTA-tris(tert-butyl ester), in each case using HATU as reagent for amide formation, to obtain the compound according to structural formula (13). Subsequently, compound (13) is fully deprotected in TFA/DCM and decoupled from the solid phase in order to obtain compound (14). Finally, the second targeting vector (9) consisting of pamidronate and squaric acid is conjugated to compound (14). The second targeting vector is previously synthesized in the same manner as shown in scheme 30.

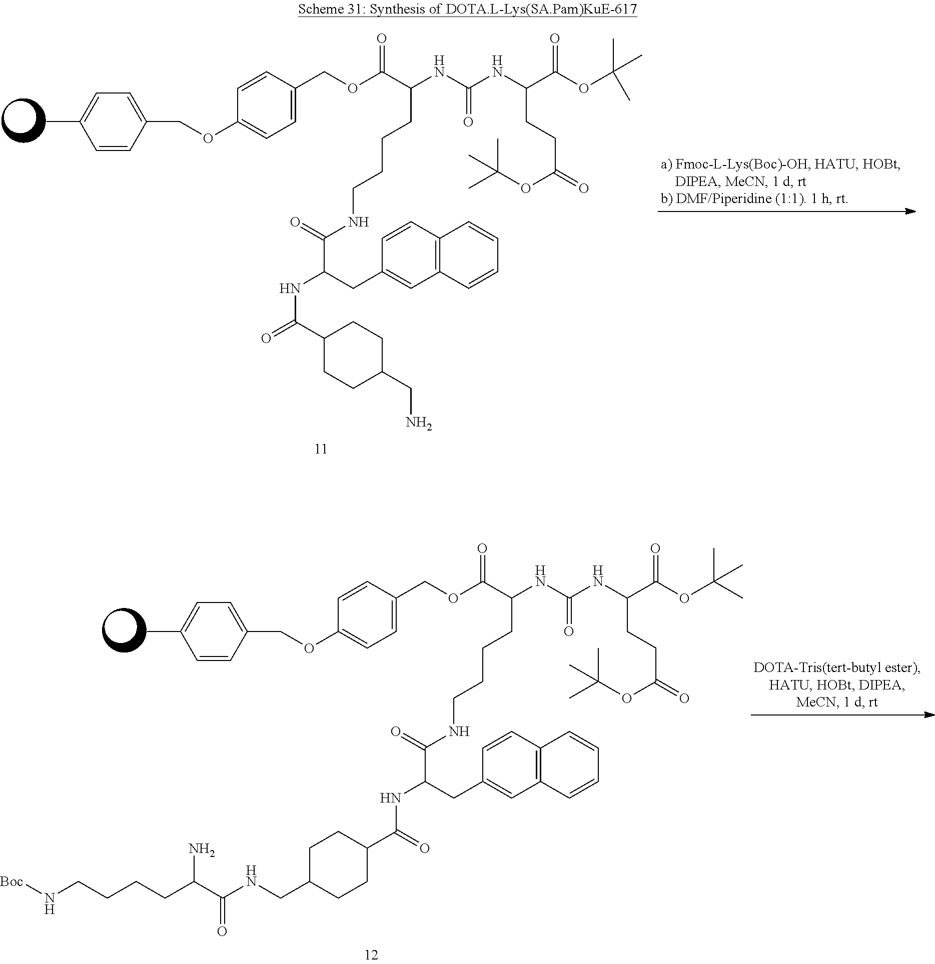 [SMILES: Cc1ccc(COc2ccc(COC(=O)C(CCCCNC(=O)C(Cc3ccc4ccccc4c3)NC(=O)C3CCC(CN)CC3)NC(=O)NC(CCC(=O)OC(C)(C)C)C(=O)OC(C)(C)C)cc2)cc1 ; Cc1ccc(COc2ccc(COC(=O)C(CCCCNC(=O)C(Cc3ccc4ccccc4c3)NC(=O)C3CCC(CNC(=O)C(N)CCCCNC(=O)OC(C)(C)C)CC3)NC(=O)NC(CCC(=O)OC(C)(C)C)C(=O)OC(C)(C)C)cc2)cc1]

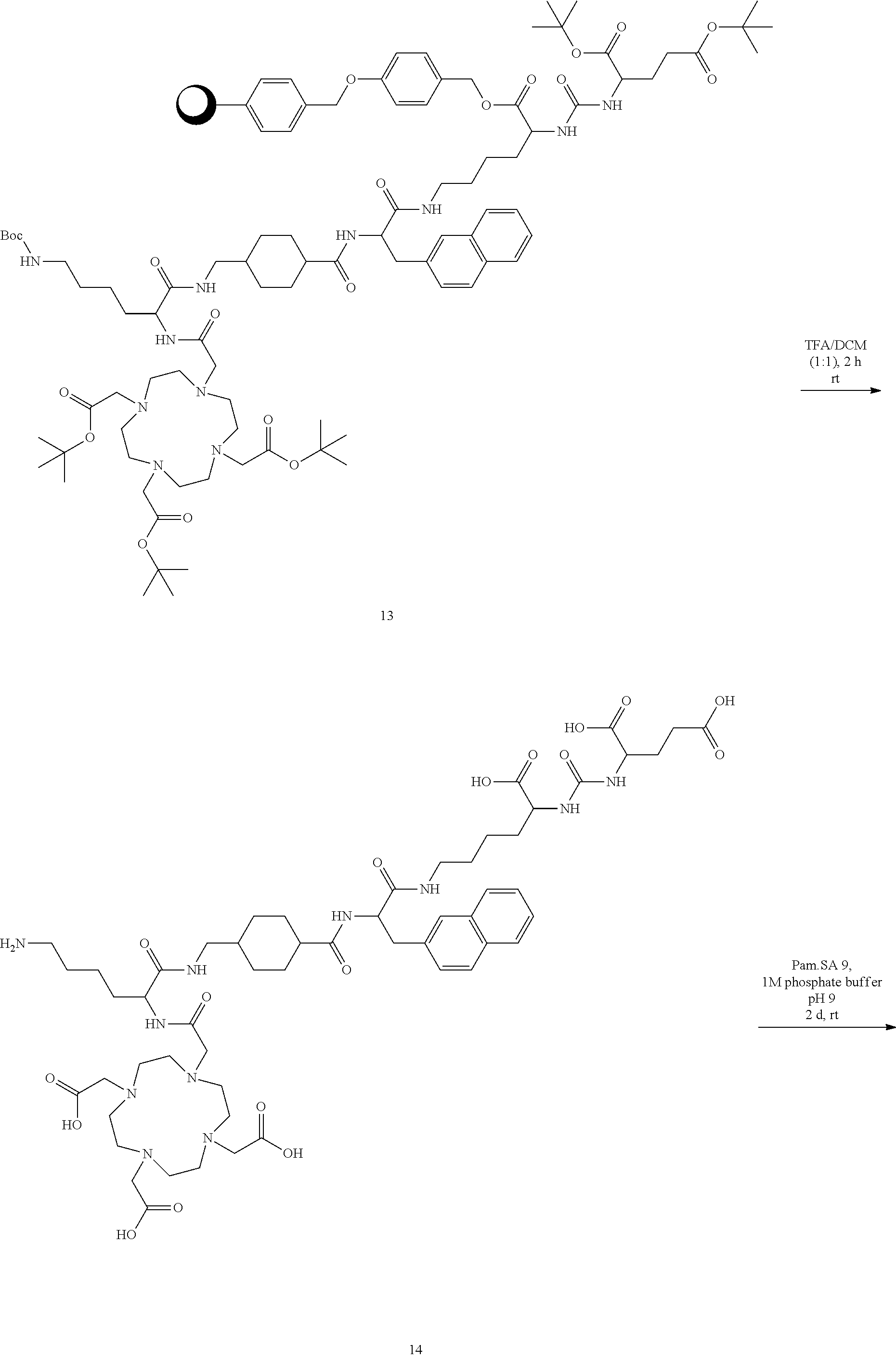 [SMILES: Cc1ccc(COc2ccc(COC(=O)C(CCCCNC(=O)C(Cc3ccc4ccccc4c3)NC(=O)C3CCC(CNC(=O)C(CCCCNC(=O)OC(C)(C)C)NC(=O)CN4CCN(CC(=O)OC(C)(C)C)CCN(CC(=O)OC(C)(C)C)CCN(CC(=O)OC(C)(C)C)CC4)CC3)NC(=O)NC(CCC(=O)OC(C)(C)C)C(=O)OC(C)(C)C)cc2)cc1 ; NCCCCC(NC(=O)CN1CCN(CC(=O)O)CCN(CC(=O)O)CCN(CC(=O)O)CC1)C(=O)NCC1CCC(C(=O)NC(Cc2ccc3ccccc3c2)C(=O)NCCCCC(NC(=O)NC(CCC(=O)O)C(=O)O)C(=O)O)CC1]

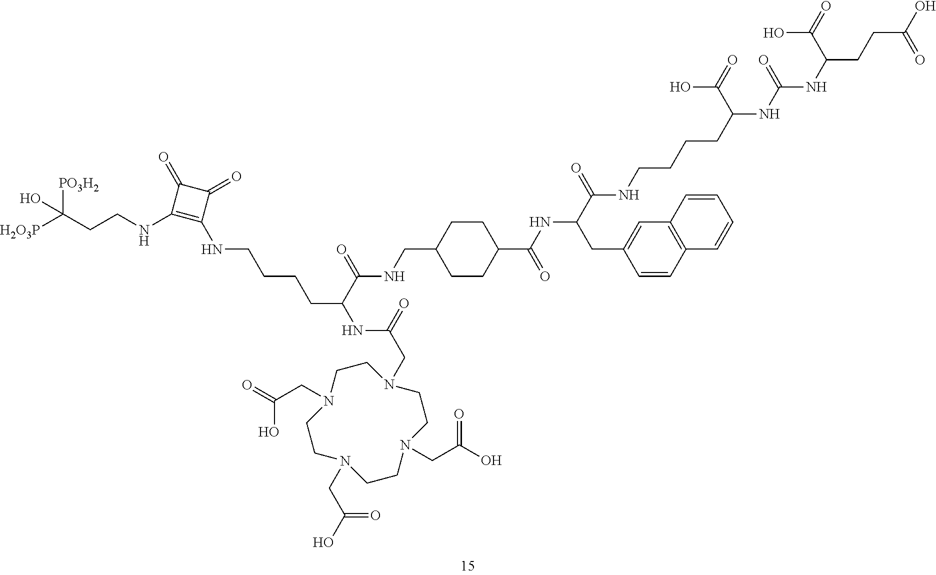

### Example 6: In Vitro Study of the Compounds NH₂.DOTAGA.KuE-617, NH₂.DOTAGA.QS.KuE and Pam.SA.DOTAGA.KuE-617

Using a cell-based assay, the affinity of the KuE targeting vector with a lipophilic linker—analogously to PSMA-617—and with a squaric acid linker was examined using the compounds NH₂.DOTAGA.KuE-617 and NH₂.DOTAGA.QS.KuE (structural formulae (8) and (10) in scheme 32).

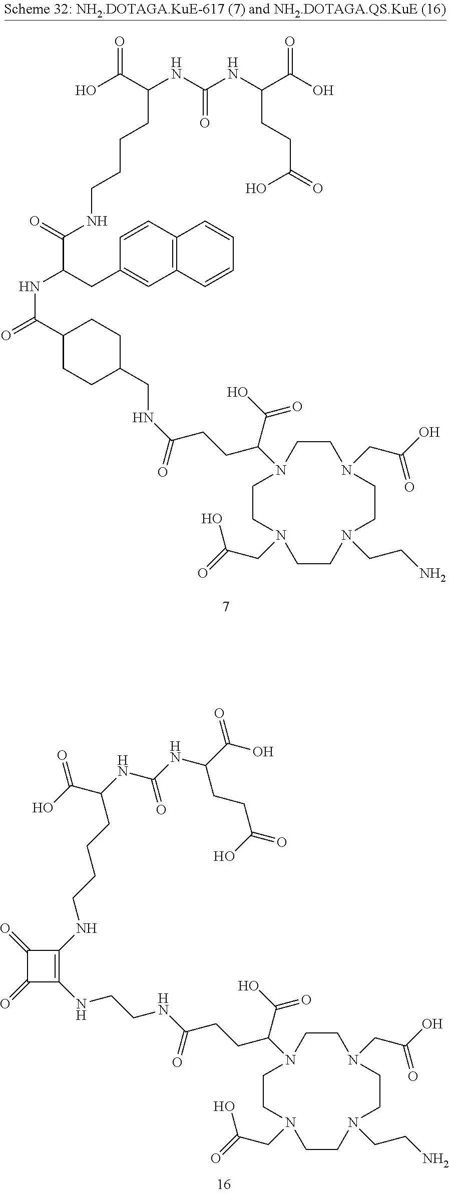 [SMILES: NCCN1CCN(CC(=O)O)CCN(C(CCC(=O)NCC2CCC(C(=O)NC(Cc3ccc4ccccc4c3)C(=O)NCCCCC(NC(=O)NC(CCC(=O)O)C(=O)O)C(=O)O)CC2)C(=O)O)CCN(CC(=O)O)CC1 ; NCCN1CCN(CC(=O)O)CCN(C(CCC(=O)NCCNc2c(NCCCCC(NC(=O)NC(CCC(=O)O)C(=O)O)C(=O)O)c(=O)c2=O)C(=O)O)CCN(CC(=O)O)CC1]

For the assay, LNCaP cells were pipetted into multiwell plates (Merck Millipore Multiscreen™). To the compounds to be analyzed, in increasing concentrations, was in each case added a defined amount or concentration of the reference compound ⁶⁸Ga[Ga]PSMA-with known K_{d}value, and the mixture was incubated in the wells with the LNCaP cells for 45 min. After washing several times, the cell-bound activity was determined. The inhibition curves obtained were used to calculate the IC₅₀ values and Kᵢ values shown in table 1.

TABLE 1
IC₅₀ values

| Compound | IC₅₀ (nM) |
|---|---|
| PSMA-617 | 15.1 ± 3.8 |
| PSMA-11 | 26.1 ± 1.2 |
| NH₂.DOTAGA.KuE-617 | 20.6 ± 3.4 |
| NH₂.DOTAGA.QS.KuE | 20.2 ± 3.5 |
| Pam.SA.DOTAGA.KuE-617 | 49.8 ± 10 |

In order to determine non-specific binding, an excess of the PSMA inhibitor 2-PMPA (2-(phosphonomethyl)pentanoic acid) was additionally added to all compounds, and they were subjected to the same LNCaP assay-as described above.

The affinities of the squaric acid-containing compound NH₂.DOTAGA.QS.KuE (16) and NH₂.DOTAGA. KuE-617 (7) are virtually the same and correspond roughly to those of the established compounds PSMA-617 and PSMA-11.

FIG. 4 illustrates the interaction of the QS.KuE group with the binding pocket of PSMA.

The complexity involved in the synthesis of the squaric acid-containing compound NH₂.DOTAGA.QS.KuE (10) is considerably lower compared to the other compounds. The use of squaric acid as a linker between the targeting vector KuE and a chelator additionally opens up a simple means of quantitatively synthesizing more complex labeling precursors with two different targeting vectors—in the present case KuE and a bisphosphonate.

Furthermore, the PSMA affinity of the final labeling precursor Pam.SA.DOTAGA.KuE-617 (10) was determined. The IC₅₀ is 49.8±10 nM.

### Example 7: Radiochemical Analysis of [¹¹⁷Lu]Lu-DOTA.L-Lys(SA.Pam)KuE-617

At a temperature of 95° C. , the labeling precursor DOTA.L-Lys(SA.Pam)KuE-617 (see scheme 17 and structural formula (15) in scheme 31) is labeled with ¹⁷⁷Lu in 1 ml of an aqueous ammonium acetate buffer solution (1 M, pH 5.5). The radiochemical yield (RCY) as a function of the amount of the labeling precursor present in the ammonium acetate buffer solution (5, and 30 nmol) is shown in FIG. 5. For an amount of the labeling precursor of ≥10 nmol, the radiochemical yield (RCY) reaches a value of ≥90% after 5 min. In contrast, with an amount of 5 nmol of labeling precursor, the yield after 5 min is only 75% and later reaches a plateau value of 85%. Radiochemical yield and purity are determined by radio thin-layer chromatography (radio-TLC) and radio high-pressure liquid chromatography (radio-HPLC). Radio thin-layer chromatography for the radiotracer [¹¹⁷Lu]Lu-DOTA.L-Lys(SA.Pam) KuE-617 gives an R_{f} of 0.0. In contrast, unbound [¹⁷⁷Lu]Lu³⁺ in citrate buffer as mobile phase gives an R_{f} of 0.8 to 1.0. In analytical radio high-pressure liquid chromatography, for the radiotracer, a retention time t_{R} of 9.8 min is measured.

FIG. 6 shows measurements of the stability of the radiotracer [¹¹⁷Lu]Lu-DOTA.L-Lys(SA.Pam)KuE-617 in phosphate-buffered saline (PBS), isotonic saline (NaCl) and human serum (HS). Even after 14 days in PBS and isotonic saline (NaCl), ≥98% of the radiotracer [¹¹⁷Lu]Lu-DOTA.L-Lys(SA.Pam)KuE-617 is present unchanged in complexed form. In human serum (HS), stability is slightly lower at 93% after 9 days, with stability remaining at 93% after 14 days.

The lipophilicity of [¹¹⁷Lu]Lu-DOTA.L-Lys(SA.Pam)KuE-617 is determined by determining the partition equilibrium of the compound in a mixture of n-octanol and PBS. Measurements for the logD_{7.4} coefficients of [¹¹⁷Lu]Lu-DOTA.L-Lys(SA.Pam)KuE-617 and [¹¹⁷Lu]Lu-PSMA-617 are presented in table 2. The results show that [¹¹⁷Lu]Lu-DOTA.L-Lys(SA.Pam) KuE-617 has virtually the same lipophilicity as PSMA-617.

TABLE 2
Radiotracer lipophilicity

| Radiotracer | LogD_{7,4} (n-octanol/PBS) |
|---|---|
| [¹¹⁷Lu]Lu-DOTA.L-Lys(SA.Pam)KuE-617 | −2.3 ± 0.12 |
| [¹⁷⁷Lu]Lu-PSMA-617 | −2.2 ± 0.20 |

### Example 8: Affinity for Hydroxyapatite (HAP)

Calcium-containing crystalline hydroxyapatite is an essential component of mammalian bones and is suitable as a model substrate for the in vitro study of bisphosphonate accumulation in healthy bone tissue and bone metastases. FIG. 7 shows measurements for the accumulation of [¹¹⁷Lu]Lu-DOTA.L-Lys(SA.Pam)KuE-617, [117Lu]Lu-PSMA-617 and [¹¹⁷Lu]Lu³⁺ on ordinary HAP and on HAP pretreated or blocked with pamidronate, with free [¹¹⁷Lu]Lu³⁺ being known to have a high affinity for HAP and serving as a reference. The proportion of the radiotracer [¹¹⁷Lu]Lu-DOTA.L-Lys(SA.Pam)KuE-617 bound to HAP is 98.2%, slightly below the value measured for free [¹¹⁷Lu]Lu³⁺ of 99.9%. In contrast, [¹¹⁷Lu]Lu-PSMA-617 is found to have an HAP-enriched fraction of only 1.2%. In order to determine selectivity, accumulation on HAP previously treated with excess pamidronate was additionally measured. This gives values of 7.3% for [¹¹⁷Lu]Lu-DOTA.L-Lys(SA.Pam)KuE-617 and 4.9% for free [¹¹⁷Lu]Lu³, which demonstrate high selectivity for HAP.

### Example 9: In Vitro Affinity for PSMA

By means of comparative radioligand assays, binding affinity for PSMA is determined for the radiotracer or labeling precursor DOTA.L-Lys(SA.Pam)KuE-617 and reference structures. Corresponding measurements for the inhibition constant Kᵢ are given in table 3. The Kᵢ-value for [^{nat}Lu]Lu-DOTA.L-Lys(SA.Pam)KuE-617 corresponds roughly to the value for the labeling precursor DOTA.L-Lys(SA.Pam) KuE-617. It can be seen from this that that complexation with lutetium does not adversely affect binding affinity for PSMA. Compared to DOTA.L-Lys.KuE-617—corresponding to structural formula (14) in scheme 31—the Kᵢ of [^{nat}Lu]Lu-DOTA.L-Lys(SA.Pam)KuE-617 is greater by a factor of about 2. It can be seen from this that the squaric acid-pamidronate group reduces affinity for PSMA.

TABLE 3
Affinity for PSMA

| Labeling precursor/compound | PSMA Kᵢ [nM] |
|---|---|
| DOTA.L-Lys(SA.Pam)KuE-617 | 53 ± 4 |
| [^{nat}Lu]Lu-DOTA.L-Lys(SA.Pam)KuE-617 | 42 ± 8 |
| DOTA.L-Lys.KuE-617 | 20 ± 3 |
| PSMA-617 | 7 ± 1 |

### Example 10: Ex Vivo Studies

For the radiotracer [¹¹⁷Lu]Lu-DOTA.L-Lys(SA.Pam)KuE-617, organ accumulation was examined in Balb/c mice with induced LNCaP tumors. The results are shown in FIG. 8. Enrichment of the radiotracer [¹¹⁷Lu]Lu-DOTA.L-Lys(SA.Pam)KuE-617 in tumor and femur is comparable, with values of 4.2±0.7% ID/g and 3.4±0.4% ID/g respectively. In contrast to tumors, accumulation in bones cannot be blocked by administering the PSMA inhibitor PMPA (2-phosphonomethylpentanedioic acid). It can be seen from this that that uptake in the bone is not caused by PSMA, since no PSMA is expressed. With a value of 17±2% ID/g, the kidneys show high accumulation of [¹¹⁷Lu]Lu-DOTA.L-Lys(SA.Pam)KuE-617, which can likewise be greatly reduced by administration of PMPA. In contrast, accumulation in the liver and spleen is not PSMA-specific. The ratio of accumulations in tumors and the blood is exceptionally high with values of 210 and 170 respectively, and suggests minor hematological side effects.

### Methods and Materials

### General

All chemicals were sourced from Sigma-Aldrich, Merck, Fluka, AlfaAesar, VWR, AcrosOrganics, TCI, Iris Biotech or Fisher Scientific and used without additional purification. Dry solvents were sourced from Merck and VWR, deuterated solvents for NMR spectra from Deutero. PSMA-617 was purchased from Hycultec. Thin-layer chromatography was performed with Merck silica gel 60 F254-coated aluminum plates. Evaluation was effected by fluorescence absorbance at λ=254 nm and staining with potassium permanganate. The radio TLCs were evaluated with a Raytest CR-35 Bio Test Imager and the AIDA software (Raytest). The ¹H and ¹³C NMR measurements were performed on a Bruker Avance III HD 300 spectrometer (300 MHz, 5 mm BBFO probe with z-gradient and ATM and BACS 60 sample changer), a Bruker Avance II 400 spectrometer (400 MHZ, 5 mm BBFO probe with z-gradient, ATM and SampleXPress 60 sample changer) and a Bruker Avance III 600 spectrometer (600 MHz, 5 mm TCI CryoProbe probe with z-gradient and ATM and SampleXPress Lite 16 sample changer). LC/MS measurements were performed on an Agilent Technologies 1220 Infinity LC system coupled to an Agilent Technologies 6130B Single Quadrupole LC/MS system. Semi-preparative HPLC purification was conducted on a Hitachi LaChrom series 7000 and with the conditions and columns mentioned in each case. For radioactive labeling experiments, [¹⁷⁷Lu]LuCl₃ in 0.04 M HCl, provided by ITM Garching, was used.

### Organic Syntheses

### Solid-Phase Synthesis of the PSMA Ligand (KuE-617 on Polystyrene Resin)

The synthesis of the glutamate-urea-lysine binding motif KuE and of the linker of the KuE-617 ligand is in accordance with established solid-phase peptide chemistry by a method proposed by Benešová et al. (Benešová, M.; Schäfer, M.; Bauder-Wüst, U.; Afshar-Oromieh, A.; Kratochwil, C.; Mier, W.; Haberkorn, U.; Kopka, K.; Eder, M. Preclinical Evaluation of a Tailor-Made DOTA-Conjugated PSMA Inhibitor with Optimized Linker Moiety for Imaging and Endoradiotherapy of Prostate Cancer. J. Nucl. Med. 2015, 56 (6), 914-920; Benešová, M.; Bauder-Wüst, U.; Schäfer, M.; Klika, K. D.; Mier, W.; Haberkorn, U.; Kopka, K.; Eder, M. Linker Modification Strategies to Control the Prostate-Specific Membrane Antigen (PSMA)-Targeting and Pharmacokinetic Properties of DOTA-Conjugated PSMA Inhibitors. J.Med.Chem. 2016, 59 (5), 1761-1775) and slightly adapted methods. Bis(tert-butyl)-L-glutamate hydrochloride (4.5 g, 15.21 mmol) and DIPEA (7.98 g, 10.5 ml, 61.74 mmol) are dissolved in dry dichloromethane (200 ml), and cooled to 0° C. Triphosgene (1.56 g, 5.26 mmol) in dichloromethane (30 ml) is added dropwise over a period of 4.5 h. After the addition is complete, the solution is stirred for a further hour. The Fmoc protecting group of Fmoc-L-lysine(Alloc)-Wang resin (1.65 g, 1.5 mmol, 0.9 mmol/g) is removed by stirring it in a piperidine/DMF solution (1:1) for 15 minutes, followed by a wash step with dichloromethane. The deprotected L-lysine (Alloc)-Wang resin is added to the previously prepared solution and stirred at room temperature overnight. The resin is washed with dichloromethane (15 ml) and used without further purification. Tetrakis(triphenylphosphine)palladium (516 mg, 0.45 mmol) and morpholine (3.92 g, 3.92 ml, 45 mmol) are dissolved in dichloromethane (12 ml) and added. The solution is stirred for 24 h in the dark. It is then washed with dichloromethane (15 ml), 1% DIPEA solution in DMF (3×13 ml) and sodium diethyldithiocarbamate trihydrate solution (15 mg/ml) in DMF (9×10.5 ml×5 minutes) to obtain resin-bound and Alloc-deprotected glutamate-urea-lysine conjugate. Fmoc-3-(2-naphthyl)-L-alanine (1.75 g, 4.00 mmol), HATU (1.52 g, 4.00 mmol), HOBt (540 mg, 4 mmol) and DIPEA (780 mg, 1.02 ml, 6.03 mmol) are dissolved in dry DMF (10 ml) and added to the resin. The solution is stirred overnight and then washed with DMF (10 ml) and dichloromethane (10 ml). To remove the Fmoc group, the resin is stirred in a piperidine/DMF solution (1:1, 3×11 ml) for 10 minutes each time and washed with DMF (10 ml) and dichloromethane (10 ml). Fmoc-4-Amc-OH (1.52 g, 4 mmol), HATU (1.52 g, 4 mmol), HOBt (540 mg, 4 mmol) and DIPEA (780 mg, 1.02 ml, 6.03 mmol) are added to the resin in dry DMF (10 ml). The solution is stirred for two days and then washed with DMF (10 ml) and dichloromethane (10 ml). To remove the Fmoc group, the reaction solution is stirred in a piperidine/DMF solution (1:1, 11 ml) for 10 min each time and washed with DMF (10 ml) and dichloromethane (10 ml) to obtain the resin-bound KuE-617 ligand.

### Pamidronate Synthesis

β-Alanine (1.5 g, 0.017 mol) and phosphoric acid (2.76 g, 0.034 mol) are dissolved in sulfolane (5.5 ml) and cooled to 0° C. Phosphorus trichloride (4.62 g, 2.95 ml, 0.034 mmol) is added dropwise. The solution is stirred at 75° C. for 3 h. Water (15 ml) is added and the mixture is stirred at 100° C. for 12 h. Finally, ethanol (15 ml) is added and, after crystallization at 0° C. for 3 days, the pamidronate product (1.48 g, 0.006 mol, 37%) is obtained as a yellow solid.

¹H NMR (300 MHZ, D₂O): [δppm]=3.34 (t, J=7.1 Hz, 2H), 2.31 (tt, J=13,7, 7.1 Hz, 2H).

¹³C NMR (400 MHz, D₂O): [δppm]=72.58; 36.14; 30.54.

¹³P NMR (121.5 MHZ, D₂O): [δppm]=17.58 (s, 2P).

MS (ESI): 236.0 [M+H]⁺, calculated for C₃H₁₁NO₇P₂: 235.07 [M]⁺.

### Synthesis of Pamidronate-Ethyl Squarate

Pamidronate (500 mg, 2.13 mmol) is dissolved in phosphate buffer (0.5 M, pH 7, 5 ml). 3,4-Diethoxycyclobut-3-ene-1,2-dione (diethyl squarate, SADE, 542 mg, 468 μl, 3.2 mmol) is added and the mixture is stirred at room temperature for 2 days. Ethanol (3 ml) is added for crystallization. The mixture is left in the freezer for 3 days to complete crystallization. The white precipitate is washed with cold ethanol and the pamidronate-ethyl squarate product (0.58 g, 1.62 mol, 76%) is obtained as a white solid.

¹H NMR (400 MHZ, D2O): [δppm]=4.79-4.62 (m, 2H), 3.31 (t, J=6.6 Hz, 2H), 2.32-2.15 (m, 2H), 1.42 (dt, J=11.7, 7.2 Hz, 3H).

³¹P NMR (162 MHZ, D2O): [δppm]=17.92 (s), 2.26 (s).

MS (ESI): 360.0 [M+H]⁺, 720.0 2[M+H]⁺, 763.0 2[M+Na]⁺, calculated for C₉H₁₅NO₁₀P₂: 359.16[M]⁺.

### Fmoc-L-Lys(Boc)-KuE-617 Resin

Fmoc-L-Lys(Boc)-OH (506 mg, 0.0011 mmol), HATU (415 mg, 0.0011 mg), HOBt (146 mg, 0.0011 mmol) and DIPEA (277 μl, 211 mg, 0.00162 mmol) are dissolved in acetonitrile (4 ml) and stirred for 30 min. The KuE-617 resin (300 mg, 0.0027 mmol, 0.09 mmol/g) is added and the mixture is stirred at room temperature for 1 day. The resin is mixed with acetonitrile (10 ml) and dichloromethane (10 ml), and kept ready for subsequent synthesis steps.

### L-Lys(Boc)-KuE-617 Resin

The Fmoc-L-Lys(Boc)-KuE-617 resin is stirred in a mixture of DMF and piperidine (1:1, 6 ml) for one hour. The Fmoc-deprotected resin is washed with DMF (10 ml) and dichloromethane (10 ml) and used in the next step without further purification.

### DOTA(tBu)3-L-Lys(Boc)-KuE-617 Resin

DOTA-tris(tert-butyl ester) (310 mg, 0.54 μmol), HATU (308 mg, 0.00081 mmol), HOBt (110 mg, 0.00081 mmol) and DIPEA (184 μl, 140 mg, 0.0011 mmol) are dissolved in acetonitrile (4 ml) and stirred for 30 min. L-Lys(Boc)-KuE-617 resin (461 mg, 0.00027 mmol, 0.9 mmol/g) is added and the mixture is stirred at room temperature for one day. The resin is washed with acetonitrile (10 ml) and dichloromethane (10 ml), and used in the next step without further purification.

### DOTA-L-Lys-KuE-617

DOTA(tBu)3-L-Lys(Boc)-KuE-617 resin (536 mg, 0.00027 mmol, 0.9 mmol/g) is stirred in a solution of TFA and dichloromethane (1:1, 4 ml). The TFA/dichloromethane solution is concentrated under reduced pressure, and the product (10.6 mg, 0.0091 mmol, 4%) is obtained as a colorless powder after semipreparative HPLC purification (column: LiChrospher 100 RP18 EC (250×10 mm) 5 m, flow rate: 5 ml/min, H₂O/MeCN+0.1% TFA, 25% MeCN isocratic, tR=10.3 min).

MS (ESI): 1172.5 [M+2H]⁺, 585.9 1/2[M+2H]⁺, 391.0 1/3[M+2H]⁺, calculated for C₅₅H₈₃N₁₁O₁₇: 1170.33 [M]⁺.

### DOTA-L-Lys(SA.Pam)-KuE-617

Compound (14) from scheme 31 (10 mg, 0.0085 mmol) and pamidronate-ethyl squarate (16 mg, 0.043 mmol) are dissolved in phosphate buffer (0.5 M, pH 9, 1 ml) and stirred for 2 days. The DOTA-L-Lys(SA.Pam)-KuE-617 product (10.56 mg, 0.0071 mmol, 84%) is obtained as a colorless powder after semipreparative HPLC purification (column: LiChrospher 100 RP18 EC (250×10 mm) 5 m, flow rate: 5 ml/min, H₂O/MeCN+0.1% TFA, 23% to 28% MeCN in 20 min, tR=8.2 min).

MS (ESI): 511.3 1/3[M+H+2Na]⁺, 520.0 [1/3M+2K]⁺, 781.0 1/2[M+2K]⁺, calculated for C₆₂H₉₂N₁₂O₂₆P₂: 1483.42 [M]⁺.

Radiolabeling of DOTA-L-Lys(SA.Pam)-KuE-617 with lutetium-177

For radioactive labeling, [¹⁷⁷Lu]LuCl₃ in 0.04 M HCl (ITG, Garching, Germany) is used. Radiolabeling is performed in 1 ml of 1 M ammonium acetate buffer at pH 5.5. Reactions are performed with different amounts of precursor (5, 10 and 30 nmol) and at 95° C. with 40-50 MBq n.c.a. lutetium-177. The reaction was monitored using radio thin-layer chromatography (TLC silica gel 60 F254 from Merck) and citrate buffer (pH 4) as mobile phase and high-pressure liquid chromatography using a HPLC 7000 Hitachi LaChrom analytical instrument (column: Merck Chromolith® RP-18e, 5-95% MeCN (0.1% TFA)/95-5% water (0.1% TFA) in 10 min). Radio thin-layer chromatography samples are measured and evaluated with the TLC Imager CR-35 Bio Test Imager from Elysia-Raytest (Straubenhardt, Germany) with AIDA software.

### In Vitro Stability Study

Stability studies of ¹⁷⁷Lu-labeled compounds are performed in human serum (HS, AB human male plasma, USA origin, Sigma-Aldrich) and phosphate-buffered saline (Sigma-Aldrich). 5 MBq of the radioactive compound is incubated in 0.5 ml of the medium for 14 days. Aliquots are taken at different times (1 h, 2 h, 5 h, 1 d, 2 d, 5 d, 7 d, 9 d and 14 d) to determine the radiochemical stability. Each measurement is carried out in triplicate.

### Determination of Lipophilicity

LogD_{7.4} of the respective compound is determined via the partition coefficient in n-octanol and PBS. The labeling solution is adjusted to pH 7.4 and 5 MBq is diluted in 700 μl of n-octanol and 700 μl of PBS. It is shaken at 1500 rpm for 2 min and then centrifuged. 400 μl of the n-octanol phase and 400 μl of the PBS phase were each transferred to a new Eppendorf tube. 3-6 μl is then pipetted onto a TLC plate and analyzed using a phosphor imager. The logD_{7.4} is calculated based on the ratio of the activities of the two phases. The measurement of each phase is also repeated twice more with the sample of higher activity, such that three logD_{7.4} values can be obtained and an average can be calculated.

### Measurement of Hydroxyapatite Affinity of ¹⁷⁷Lu-Labeled Compounds

Hydroxyapatite (20 mg) is incubated in saline (1 ml) for 24 h. 50 μl of the radiotracer [¹⁷⁷Lu]Lu-DOTA-L-Lys(SA.Pam)-KuE-617 (5 MBq) or [¹⁷⁷Lu]Lu-PSMA-617 (5 MBq) is added. Each suspension is vortexed with a vortex mixer for 20 s and incubated at room temperature for 1 h. Each suspension is then passed through a filter (CHROMAFIL® Xtra PTFE-45/13), and the supernatant is washed with water (500 μl). The radioactivity of the liquids and HAP-containing supernatants obtained is measured in each case with a curiemeter (Isomed 2010 activimeter, MED Nuclear-Medizintechnik Dresden GmbH). The binding of [¹⁷⁷Lu]Lu-DOTA-L-Lys(SA.Pam)-KuE-617 and [¹⁷⁷Lu]Lu-PSMA-617 is determined as a percentage of the activity absorbed on HAP. As a reference, the HAP binding of free Lu-177 is measured in an analogous manner. Comparative measurements are carried out on blocked hydroxyapatite in an analogous manner. For this purpose, HAP (20 mg) in saline solution (1 ml) is incubated with pamidronate (100 mg) and the respective activities of [¹⁷⁷Lu]Lu-DOTA-L-Lys(SA.Pam)-KuE-617 and free Lu-177 are determined.

### In Vitro Study of PSMA Binding Affinity

Non-active (cold) [natLu]Lu complexes are prepared by shaking a solution containing the labeling precursor DOTA-L-Lys(SA.Pam)-KuE-617 (371 μl, 1 mg/ml, 250 nmol) with LuCl₃ (129 μl, 1 mg/ml, 375 nmol, metal to labeling precursor ratio 1.5:1) in 1 M ammonium acetate buffer at 95° C. for 2 hours. Complex formation is monitored by ESI-LC/MS.

PSMA binding affinity is determined by the competitive radioligand assay described by Benešová et al. (Benešová, M.; Schaefer, M.; Bauder-Wüst, U.; Afshar-Oromieh, A.; Kratochwil, C.; Mier, W.; Haberkorn, U.; Kopka, K.; Eder, M. Preclinical Evaluation of a Tailor-Made DOTA-Conjugated PSMA Inhibitor with Optimized Linker Moiety for Imaging and Endoradiotherapy of Prostate Cancer. J. Nucl. Med. 2015, 56 (6), 914-920.). For this purpose, PSMA-positive LNCaP cells from Sigma-Aldrich in RPMI 1640 (Thermo Fisher Scientific) supplemented with 10% fetal bovine serum (Thermo Fisher Scientific), 100 μg/ml streptomycin and 100 units/ml penicillin are cultured at 37° C. in 5% CO₂. The LNCaP cells are incubated with increasing concentrations of solutions containing the labeling precursors in the presence of 0.75 nM [⁶⁸Ga]Ga-PSMA-10 for 45 min. Free radioactivity is removed by several washes with ice-cold PBS. The samples obtained are measured in a y counter (2480 WIZARD2 Automatic Gamma Counter, PerkinElmer). The measurement data are evaluated in GraphPad Prism 9 using non-linear regression.

### Ex Vivo Studies

All animal experiments were approved by the ethics committee of the state of Rhineland-Palatinate (according to § 8 para. 1 Tierschutzgesetz [Animal Protection Act], Landesuntersuchungsamt [State Investigation Office]) and carried out in accordance with the relevant federal laws and institutional guidelines (approval no. 23 177-07/G 21-1-022). 6- to 8-week-old BALB/cAnNRj males (Janvier Labs) were inoculated subcutaneously with 5×10⁶ LNCaP cells in 200 μl 1:1 (v/v) Matrigel/PBS (Corning®). Measurements were conducted after the tumor reached a volume of about 100 cm³. Before intravenous injection of 0.5 nmol [¹⁷⁷Lu]Lu-DOTA-L-Lys(SA.Pam)-KuE-617, the LNCaP tumor-bearing mice were anesthetized with 2% isoflurane. The specific activity was about 3 MBq/nmol. PSMA selectivity was examined by coinjecting 1.5 mmol of PMPA per mouse. The animals were sacrificed 24 h p.i. The organs were collected and weighed. Radioactivity was measured and calculated as a decay-corrected percentage of injected dose per gram of tissue mass % ID/g.

1. A labeling precursor for complexing radioactive isotopes having the structure 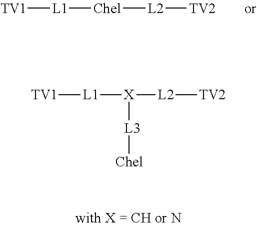
in which
a first targeting vector TV1 is selected from the group of PSMA inhibitors comprising 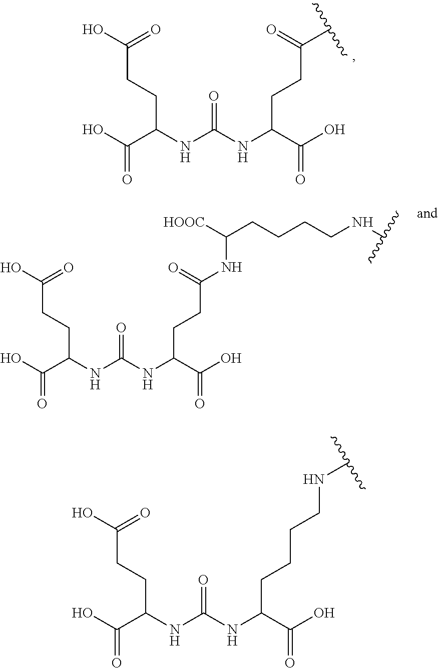
a second targeting vector TV2 is selected from the group of bisphosphonates comprising 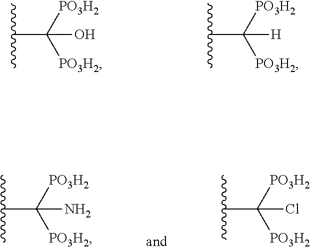
a first linker L1 has a structure selected from 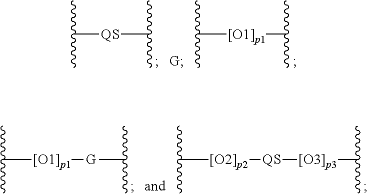
in which
G is 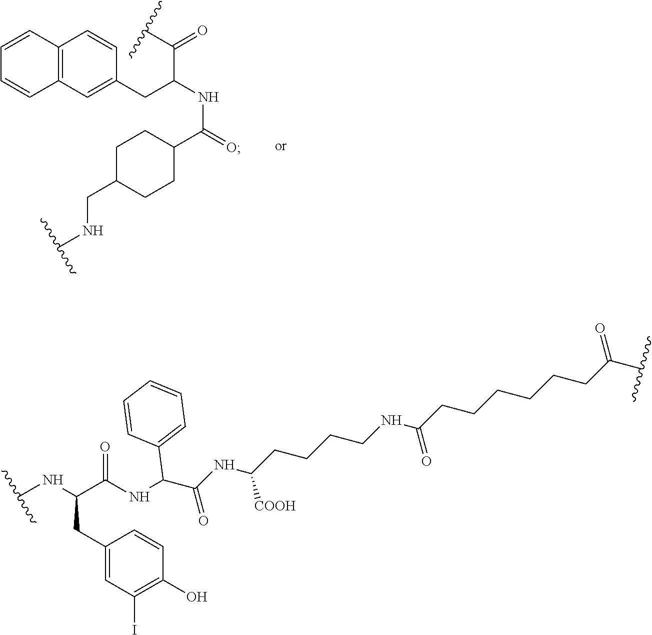 [SMILES: *NCC1CCC(C(=O)NC(Cc2ccc3ccccc3c2)C(*)=O)CC1 ; *N[C@H](Cc1ccc(O)c(I)c1)C(=O)NC(C(=O)N[C@H](CCCCNC(=O)CCCCCCC(*)=O)C(=O)O)c1ccccc1]
O1, O2 and O3 are independently selected from the group comprising amide radicals, carboxamide radicals, phosphinate radicals, alkyl radicals, triazole radicals, thiourea radicals, ethylene radicals, maleimide radicals, —(CH₂)—, —(CH₂CH₂O)—, —CH₂—CH(COOH)—NH— and —(CH₂)_{q}NH— with q=1, 2, 3, 4, 5, 6, 7,8,9 or 10;
p1, p2 and p3 are independently selected from the set {0, 1, 2, 3, 4, 5, 6, 7, 8, 9, 10, 11, 12, 13, 14, 15, 16, 17, 18 , 19, 20};
a second linker L2 has a structure selected from 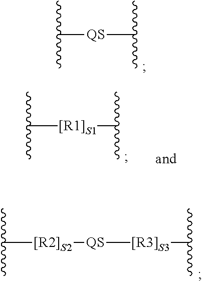
in which R1, R2 and R3 are independently selected from the group comprising amide radicals, carboxamide radicals, phosphinate radicals, alkyl radicals, triazole radicals, thiourea radicals, ethylene radicals, maleimide radicals, furan radicals, azole radicals, oxazole radicals, thiophene radicals, thiazole radicals, azine radicals, thiazine radicals, naphthalene radicals, quinoline radicals, pyrrole radicals, imidazole radicals, pyrazole radicals, tetrazole radicals, thiadiazole radicals, oxadiazole radicals, pyridine radicals, pyrimidine radicals, triazine radicals, tetrazine radicals, thiazine radicals, oxazine radicals, naphthalene radicals, chromene radicals or thiochromene radicals, —(CH₂)—, —(CH₂CH₂O)—, —CH₂—CH(COOH)—NH— and —(CH₂)_{q}NH— with q=1, 2, 3, 4, 5,6, 7, 8,9 or 10;
s1, s2 and s3 are independently selected from the set {0, 1, 2, 3, 4, 5, 6, 7, 8, 9, 10, 11, 12, 13, 14, 15, 16, 17, 18 , 19, 20};
a third linker L3 has a structure selected from 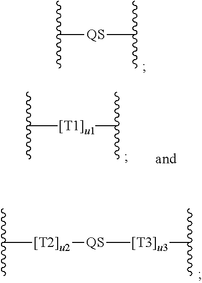
in which
T1, T2 and T3 are independently selected from the group comprising amide radicals, carboxamide radicals, phosphinate radicals, alkyl radicals, triazole radicals, thiourea radicals, ethylene radicals, maleimide radicals, —(CH₂)—, —(CH₂CH₂O)—, —CH₂—CH(COOH)—NH— and —(CH₂)ᵥNH— with v=1, 2, 3,4, 5, 6,7,8,9 or 10;
u1, u2 and u3 are independently selected from the set {0, 1, 2, 3, 4, 5, 6, 7, 8, 9, 10, 11, 12, 13, 14, 15, 16, 17, 18 , 19, 20};
QS is a squaric acid radical; 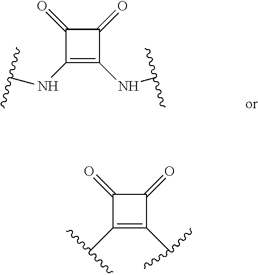 [SMILES: *Nc1c(N*)c(=O)c1=O ; *c1c(*)c(=O)c1=O]
and
Chel is a chelator selected from the group comprising H₄pypa, ethylenediaminetetraacetate (EDTA), diethylenetriaminepenta(methylenephosphonic acid) (EDTMP) diethylenetriaminepentaacetate (DTPA) and derivatives thereof, dodeca-1,4,7,10-tetraamine tetraacetate (DOTA), 2-(1,4,7,10-tetraazacyclododecane-4,7,10)pentanedioic acid (DOTAGA) and other derivatives, trideca-1,4,7,10-tetraamine tetraacetate (TRITA), tetradeca-1,4,8,11-tetraamine tetraacetate (TETA) and derivatives thereof, nona-1,4,7-triamine triacetate (NOTA) and derivatives thereof, triazacyclononanephosphinic acid (TRAP), 1,4,7-triazacyclononane-1,4-bis[methylene(hydroxymethyl)phosphinic acid]-7-[methylene(2-carboxyethyl)phosphinic acid] (NOPO), pentadeca-1,4,7,10,13-pentaamine pentaacetate (PEPA), hexadeca-1,4,7,10,13,16-hexaamine hexaacetate (HEHA) and derivatives thereof, hydroxybenzylethylenediamine (HBED) and derivatives thereof, DEDPA and derivatives thereof, deferoxamine (DFO) and derivatives thereof, trishydroxypyridinone (THP) and derivatives thereof, tetraazacyclodecanephosphinic acid (TEAP) and derivatives thereof, 6-amino-6-methylperhydro-1,4-diazepine N,N,N′,N′-tetraacetate (AAZTA) and derivatives; 1-N-(4-aminobenzyl)-3,6,10,13,16,19-hexaazabicyclo[6.6.6]eicosane-1,8-diamine (SarAr) and salts thereof, 1,8-diamino-3,6,10,13,16,19-hexaazabicyclo[6.6.6]eicosane ((NH₂)₂SAR) and salts and derivatives thereof, aminothiols and derivatives thereof.

2. The labeling precursor as claimed in claim 1, wherein the chelator Chel is DOTA, H₄pypa, DATA or DOTAGA.

3. The labeling precursor as claimed in claim 1, wherein the second linker L2 comprises at least one radical selected from

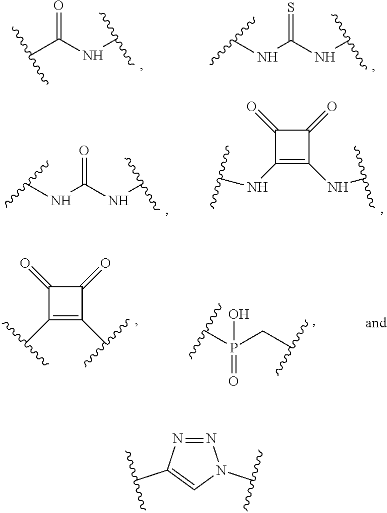

4. The labeling precursor as claimed in claim 3, wherein the second linker L2 comprises at least one squaric acid radical

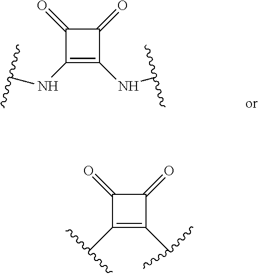 [SMILES: *Nc1c(N*)c(=O)c1=O ; *c1c(*)c(=O)c1=O]

5. The labeling precursor as claimed in claim 3, wherein the second linker L2 comprises at least one radical selected from

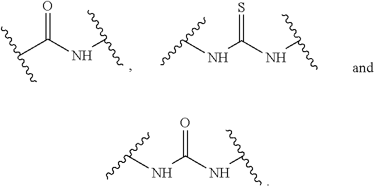 [SMILES: *NC(*)=O ; *NC(=S)N* ; *NC(=O)N*]

6. The labeling precursor as claimed in claim 1, wherein the second linker L2 comprises at least one imidazole radical

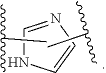

7. The labeling precursor as claimed in claim 1, wherein two or three of the linkers L1, L2 and L3 are the same.

8. A radiotracer comprising a labeling precursor as claimed in claim 1 and a radioactive isotope selected from the group comprising ⁴⁴Sc, ⁴⁷Sc, ⁵⁵Co, ⁶²Cu, ⁶⁴Cu, ⁶⁷Cu, ⁶⁶Ga, ⁶⁷Ga, ⁶⁸Ga, ⁸⁹Zr, ⁸⁶Y, ⁹⁰Y, ⁸⁹Zr, ⁹⁰Nb, ^{99m}Tc, ¹¹¹In, ¹³⁵Sm, ¹⁴⁰Pr, ¹⁵⁹Gd, ¹⁴⁹Tb, ¹⁶⁰Tb, ¹⁶¹Tb, ¹⁶⁵Er, ¹⁶⁶Dy, ¹⁶⁶Ho, ¹⁷⁵Yb, ¹⁷⁷Lu, ¹⁸⁶Re, ¹⁸⁸Re, ²¹¹ At, ²¹²Pb, ²¹³Bi, ²²⁵ Ac and ²³²Th.

9. The radiotracer as claimed in claim 8, wherein the radioactive isotope is ⁶⁸Ga, ¹⁷⁷Lu or ²²⁵ Ac.

10. A labeling precursor as claimed in claim 8, wherein the NOTA derivative is 1,4,7-triazacyclononane, 1-glutaric acid,4,7-acetate (NOTAGA), the DEDPA derivative is 1,2-[[6-(carboxylate)pyridin-2-yl]methylamine]ethane (H₂DEDPA), the THP derivative is YM103, and the AAZTA derivatives is (6-pentanoic acid)-6-(amino)methyl-1,4-diazepine triacetate-(DATA).
